(19) United States
(12) Patent Application Publication
(10) Pub. No.: US 2004/0005302 A1
(43) Pub. Date: Jan. 8, 2004

(54) Encapsulated cells to elicit immune responses

(76) Inventor: Gonzalo Hortelano, Burlington (CA)

Correspondence Address: FISH & RICHARDSON, PC, 12390 EL CAMINO REAL, SAN DIEGO, CA, 92130-2081, US

(21) Appl. No.: 10/428,519

(22) Filed: Apr. 30, 2003

Related U.S. Application Data
(60) Provisional application No. 60/377,039, filed on Apr. 30, 2002.

(51) Int. Cl. A61K 48/00

(52) U.S. Cl. 424/93.21; 514/44

(57) ABSTRACT

The present invention provides a novel approach to vaccination and/or treatment using encapsulated cells for eliciting immune responses. More specifically, the present invention is to provide a method or process for inducing an immune response in a host, wherein the process comprises the steps of: enclosing genetically engineered antigen-producing cells comprising one or more transgene, into an immunoisolating implantable device to provide encapsulated antigen-producing cells; introducing the encapsulated antigen-producing cells in the host; production of the transgene antigen product; bi-directional passage of the produced antigen product through the pores of the microcapsules, with preclusion of the passage of the antigen-producing cells therethrough; delivery of a continuous infusion of antigen to the host; activation of the immune system in response to the antigen. The present invention additionally provides a method of vaccinating or immunizing a host, a vaccine against a transgene antigen, and the use of immunoisolating implantable devices comprising genetically engineered antigen-producing cells.

### CROSS-REFERENCE TO RELATED APPLICATIONS

This application claims priority to earlier filed U.S. Patent Application Ser. No. 60/377,039 filed Apr. 30, 2002. This application is explicitly incorporated herein by reference in its entirety and for all purposes.

### TECHNICAL FIELD

This invention relates to encapsulated cells for eliciting immune responses. More particularly, it relates to a novel approach to immunization and therapy.

### BACKGROUND OF THE INVENTION

Successful vaccination requires the identification of a relevant immunogen, knowledge of minimal effective dose, an efficacious schedule and route of administration in order to induce durable immunity. Although conventional processes for vaccine fabrication utilize culture and immunogen fractionation to produce sufficient amounts of immunogens, many prospective antigens cannot be produced readily by these methods. To overcome this deficiency, recombinant nucleic acid technologies have been explored to produce immunogens. However, many recombinant immunogens often lack critical post-translational modifications normally occurring in wild type antigens. The downstream purification process can affect immunogen structure, which together can reduce efficacy. Viral and bacterial vectors have been used as heterologous expression systems, although the host often develops neutralizing antibodies against the vector, thereby limiting its replication and immunogen expression, following secondary vaccination. Genetic DNA vaccines can result in protection in various murine disease models as TB, hepatitis, malaria etc, but have not shown the same results in others animal models. Given these difficulties, there is a need to develop immunogen delivery systems that are capable of ensuring the appropriate supply of authentic immunogens to establish long-lasting vaccine efficacy.

Gene therapy holds great promise for the treatment of many diseases, where vectors that are highly efficient in delivering genes to a specific tissue, without generating immune responses, are of great interest for gene therapy. The immune system is important in preventing sustained expression of foreign genes since the cells infected with these vectors may be rapidly eliminated. In contrast, for cancer immunotherapy and vaccine development for infectious diseases, a strong immune response in the host, namely a humoral and cellular response, against the product of the delivered transgene is desired.

A variety of approaches have been devised to site-specifically augment the genomic sequence of a disease causing gene, or the expression of its corresponding product, in a universal cell line at physiological levels suitable for implantation into any patient with the disease. The optimization of this kind of therapy requires accurate definitions of the immune response against the secreted transgene product to evaluate the viability and the efficiency for treatment of a genetic disease, such as hemophilia B.

Hemophilia B is an X-linked bleeding disorder caused by a defect or deficiency in blood coagulation factor IX (Brownlee G. G., British Medical Bulletin, 51(1):91-105, 1995). Gene therapy may be a future alternative to current therapies based on the regular infusion of plasma-derived or recombinant products (Lozier J. N., and Brinkhous K. M., JAMA 270(1):47-51, 1994; Walker I., Hemophilia Today, Summer, 1997). Current treatment for hemophilia B consists of intravenous infusion of either plasma derived or recombinant clotting human factor IX (hFIX) concentrates. Because of the safety and expense of replacement products, infusions are generally given only in response to bleeds and not prophylactically. Since relatively low amounts of protein expression would be sufficient for the amelioration of severe disease, gene therapy which could provide sustained expression of a functional clotting factor in circulation, would prevent bleeds. A major goal of gene therapy for hemophilia B relates to achieving the long-term expression of therapeutic levels of hFIX without generating an immune response against the transgene product. This is especially important, because antibodies against factor IX, for instance, would preclude its use in hemophilia B and would subject the patient to the risk of anaphylaxis and nephrotic/nephritic syndromes with further exposure to factor IX protein concentrates.

Immunocompetent C57BL/6 mice are generally considered not to elicit immune responses against human Factor IX (hFIX). Therefore, hFIX is considered a weak antigen for C57BL/6 mice. However, although the generation of an immune response against hFIX is not desired for treatment in hemophilia patients, it would be desirable that antigens, like hFIX, which are weakly immunogenic antigens, be able to produce an immune response when necessary. That is to say, there are various antigens that are weakly immunogenic antigens, wherein such antigens either can not elicit an immune response, or produce a very weak, unsustained immune response.

Accordingly, there is a need to provide a means for generation of an immune response, and more preferably a complete and sustained immune response, against antigens that are generally incapable of eliciting an immune response.

It would be desirable to provide a method of inducing an immune response in a host against an antigen. And more particularly, it would be desirable to provide a means for inducing an immune response against antigens that are weakly immunogenic, or can not conventionally generate immune responses in a particular host.

It would also be desirable to provide a means of vaccinating a host against antigens that are weakly immunogenic, or can not generally generate immune responses in a host.

It has been shown in the prior art that microcapsules may be used to deliver a therapeutic product to a host. However, it would be preferable to use encapsulated genetically engineered cells to produce an antigen product, and not simply encase a product for secretion. Therefore, converse to the prior art where an antigen product may be encapsulated for secretion, it would be advantageous to encapsulate cells capable of producing antigen allowing for the continuous and sustained production and infusion of antigen in vivo to a host, thereby providing a continuous internal supply of antigen. Furthermore, the presence of encapsulated cells might further increase the immunogenecity of the antigen.

In addition, it would be advantageous to produce a continuous and sustained infusion of antigen, any antigens, even weak antigens, which would be suitable for the induction of immune responses, and allow for the vaccination of a host against such antigens, even against weak antigens, and additionally against multiple antigens. Conventionally, immune responses are not generally generated or effective for weak antigens, and accordingly, conventional vaccination techniques may not be suitable for antigens that are immunogenically weak, where the elicitation of an immune response in a host is either difficult or impossible. Accordingly, it would be desirable to provide a new means of vaccination against various antigens, in particular for antigens for which there is no conventional vaccination technique. It would also be desirable that a new means of vaccination would allow for the induction of a complete and sustained immune response, for example, humoral and cellular immune responses, against any antigen, even antigens that conventionally could not produce such immune responses.

Conventional immunization protocols can not often elicit a suitable immune response, particularly a cellular immune response, such as when the selected antigen is too similar to an endogenous product of the host. Accordingly, if the host would see such an antigen as ‘self and not ‘foreign’, it would not result in a strong or complete immune response against the antigen. Thus, for weakly immunogenic antigens, a strong immune response would not be generally elicited. Accordingly, it would be desirable to provide a process or method which would elicit strong and sustained immune responses to any antigen, and more preferably, allow for the strong, sustained and complete activation of the immune system against the antigen.

There are examples in the literature indicating that the delivery of foreign transgenes from cells transplanted in immunoisolation devices lead to transient delivery, due to immune responses to the foreign transgene (Carr-Brendel V. E., et al., Methods Mol Biol. 63:373-387, 1997; Brauker J., et al., Hum Gene Ther. 9(6):879-888, 1998). In contrast, delivery of an endogenous transgenes is sustained in vivo. This is due to the fact that the host does not recognise the transgene as foreign. Indeed, encapsulated C2C12 myoblasts engineered to secrete human erythropoietin did not yield sustained transgene delivery in immunocompetent mice (Regulier E., et al., Gene Ther. 5(8):1014-1022, 1998). In contrast, the levels of mouse erythropoietin following the same treatment were sustained (Regulier E., et al., 1998). These findings indicate that foreign transgenes proteins cannot be delivered in a sustained fashion, even from the protection of immunoisolation devices.

The results of Hortelano et al (Blood, 1996; Hum Gene Ther., 1999; and Haemophilia, 2002), as well as those of others (Blau, H. M., et al., Trends in Genetics, 9:269-274, 1993) support the view that myoblasts are suitable cells for delivering transgenes. Myoblast biopsies are relatively easy to obtain (Blau, H. M., et al., Proceedings of the National Academy of Sciences, USA 78:10892-10895), and myoblasts support transgene expression very efficiently (Blau, H. M., et al., 1993). A characteristic that makes myoblasts particularly suitable for encapsulation is their capacity to differentiate into myotubes. As opposed to proliferative cell lines, differentiation limits the myoblast growth within the microcapsule space, allowing for the long-term viability of the enclosed cells (Carr-Brendel V. E., et al., 1997). Furthermore, myotubes are very stable structures that can contribute to the long-term effectiveness of a gene therapy protocol.

Mouse C2C12 myoblasts have been used to secrete human factor IX (Dai, Y., et al., Proceedings of the National Academy of Sciences, USA 89:10892-10895, 1992; Yao, S., and K. Kurachi, Proceedings of the National Academy of Sciences, USA 89: 3357-3361; 1992). We have demonstrated that microcapsules containing recombinant allogeneic myoblasts can deliver human factor IX in mice, as shown by Hortelano et al (Blood, 1996; Hum Gene Ther., 1999; and Haemophilia, 2002). While direct detection of factor IX (ELISA) was observed for at least two weeks (Hortelano, G., Blood, 1996), there was indirect evidence for continuous delivery of factor IX in vivo for over 7 months. Therefore, noting that microcapsules have a protective effect. Accordingly, the encapsulated myoblasts remained viable in vivo for at least 7 months, as determined after their retrieval from the mice (Hortelano, G., Blood, 1996).

We have shown that mice implanted with encapsulated cells secreting human factor IX develop a high titre of antibodies against the foreign transgene (Hortelano, G., Haemophilia, 2001). Furthermore, antibodies are maintained at a high level for at least 7 months. Interestingly, it should be noted that C57BL/6 mice are generally thought not to develop antibodies against human factor IX. Although these finding suggest the mounting of a humoral immune response, by the secretion of antibodies, there is no indication or evidence relating to the mounting of a cellular immune response. Accordingly, it would be advantageous to provide a process for the induction and activation of a complete immune response, wherein both arms of the immune system are activated.

### SUMMARY OF THE INVENTION

An object of the present invention is to provide a novel approach to immunization and therapy using encapsulated cells for eliciting immune responses and/or delivering therapeutic products in vivo.

Another object of the present invention is to provide a method for providing constant, long term stimulation of a host immune system.

An object of the present invention is to provide a process for inducing an immune response in a host, wherein the process comprises the steps of: enclosing genetically engineered antigen-producing cells comprising one or more transgene encoding antigen, into an immunoisolating implantable device to provide encapsulated antigen-producing cells; implanting the encapsulated antigen-producing cells in the host; production of the transgene antigen product; bi-directional passage of the produced antigen product through the pores of the microcapsules, with preclusion of the passage of the antigen-producing cells therethrough; delivery of a continuous infusion of antigen to the host; activation of the immune system in response to the antigen.

Another object of the present invention is to provide for the use of immunoisolating implantable devices enclosing antigen-producing cells genetically engineered to contain genetic material coding for one or more antigen(s) to immunize a host against said antigen(s). In an alternative embodiment, antigen may be any antigen, or weakly immunogenic antigen, such FIX or hFIX.

It is understood by one skilled in the art, that various appropriate antigen-producing cells may be used in accordance with the present invention. In accordance with an embodiment of the present invention, the recombinant antigen-producing cells comprise a myoblast cell, or C2C12 mouse myoblast cells, or JPW01 myoblast cells or mammalian cells or human cells. According to yet another embodiment of the present invention, the mammalian or human cells may be cancer cells.

Accordingly, the term host, when referred to herein refers to any host, and may comprise a mammalian host, and may comprise a human host.

It may also be noted that the encapsulated antigen-producing cells may be an autologous, syngeneic, allogeneic or xenogeneic cell with respect to said host.

More specifically, in accordance with an embodiment of the present invention it is understood that not all myoblasts would be suitable for eliciting immune responses in a host, at least not without additional genetic engineering. Accordingly, it can be noted that the more the encapsulated cell is distant in origin from the host, that is to say, from a different species or with a certain degree of transformation, the higher the immune response that would be expected.

In accordance with an embodiment of the present invention, the transgene antigen may be a weakly immunogenic antigen. In an embodiment, the transgene antigen product may be coagulation factor IX (FIX), or human factor IX (hFIX). In addition, the genetically engineered antigen-producing cells may produce more than one transgene antigen product, wherein the antigens may be different antigens. That is to say, the genetically engineered antigen-producing cells may comprise more than one different transgene which produce different transgene antigen products, or more than one different genetically engineered antigen-producing cells may be encapsulated in a immunoisolating implantable device, or biocompatible microcapsule, so as to produce different transgene antigen products.

According to one embodiment of the present invention, antigen-producing cells of the present invention are genetically engineered to contain genetic material, herein also referred to as one ore more transgene(s), coding for one or more antigens and/or therapeutic products of interest.

According to an aspect of the present invention, the immunoisolating implantable device may comprise biocompatible microcapsules, comprising a hydrogel material, or may comprise alginate-polylysine microcapsules, or may comprise any implantable cell immunoisolating device such as biocompatible microcapsule suitable for the encapsulation of genetically engineered antigen-producing cells in a host. In accordance with alternative aspects of the present invention, the immunoisolating implantable device, or simply biocompatible microcapsules, comprise pores, wherein the pores allow for the bi-directional free flow of the produced antigen therethrough, but do not allow the free flow of antigen-producing cells. For example, the pore size may have dimensions that that allow the bi-directional free-flow of molecules with a molecular weight up to that of the produced antigen. For example, if the antigen produced by the antigen-producing cells for secretion is hFIX, the dimensions of the pores of the microcapsule would allow for molecules with a molecular weight of hFIX, or less, (for example, a molecular weight cut off at 300,000 daltons) to flow therethrough the pores, but not for the flow of the antigen-producing cells.

In accordance with an embodiment of the present invention, the encapsulated antigen-producing cells may be implanted by injection, for example, by intraperitoneal injection, subcutaneous injection, or intramuscular injection, or any other form of administration, that would allow for the introduction of viable microencapsulated recombinant antigen-producing cells to a host.

In accordance with the present invention, the encapsulated genetically engineered antigen-producing cells allow for the production and secretion of the desired transgene antigen product so as to provide a continuous infusion of antigen, thereby providing a prophylactic or therapeutic supply of antigen to said host. More particularly, the encapsulated antigen-producing cells produce an in vivo continuous supply of active antigen to said host, that allow for the induction of immune responses. Alternatively, the encapsulated genetically engineered cells of the present invention maybe adapted to provide one or more therapeutic products in vivo for treating one or more disease(s) and/or condition(s) afflicting a host.

The present invention also provides encapsulated antigen-producing cells adapted to provide one or more therapeutic products in vivo for the treatment of a chronic infectious and/or neoplastic disease and/or condition. It is understood by one of skill in the art that the present invention may be employed to treat and/or protect against any number of chronic infectious and/or neoplastic diseases and/or conditions, including but not limited to cancer or HIV.

Accordingly, the present invention provides a means of inducing immune responses in a host. More specifically, the encapsulated genetically engineered antigen-producing cells of the present invention allow for the sustained activation of the immune system, wherein both humoral and cellular immune responses may be activated. Therefore, the present invention allows for the mounting of a complete and sustained immune response to various antigens and, in onwe aspect, to antigens for which immune responses are generally not elicited. As referred to herein, a complete immune response refers to the activation of both cellular and humoral immune responses. More specifically, the present invention allows for the induction of a complete immune response against various antigens, even weakly immunogenic antigens, that is, antigens that do not generally induce an immune response. For example, the present invention may be used to immunize a host against weak antigens, against tumor antigens, or against infectious diseases, but not limited to those specific to certain disorders. More specifically, in accordance with the teachings of the present invention, the use of specific transgene antigens for the production of a continuous supply of an antigen product in a host, by encapsulated genetically engineered antigen-producing cells allows for the induction of a complete and sustained immune response against the transgene antigen. That is to say, antibodies against the transgene antigen are produced, and cytotoxic T lymphocytes are stimulated.

In accordance with a one use of the present invention, there is provided a method of vaccinating a host against an antigen. That is to say, in accordance with an embodiment of the present invention, the sustained and continuous production and infusion of antigen in vivo to a host, by means of encapsulated genetically engineered antigen-producing cells allows for the effective mounting of a complete immune response against the produced and secreted transgene antigen, therefore providing a method of vaccinating a host against infectious agents and/or cells comprising the specific antigen. Accordingly, the present invention provides a method for vaccinating a host with a weakly immunogenic antigen, that is, antigens that do not generally elicit an immune response.

In accordance with an embodiment of the present invention, there is provided a method of vaccinating a host, wherein the method comprises: introducing an immunoisolating implantable device enclosing antigen-producing cells comprising one or more transgenes encoding antigen, into said host; production of transgene antigen product by the antigen-producing cells; bidirectional passage of the produced antigen product through the pores of the implantable device, with preclusion of the passage of the antigen-producing cells therethrough; delivery of a continuous infusion of antigen to the host; sustained activation of the immune system in response to the antigen. In a one embodiment of the present invention, the immune response accordingly activated is a complete and sustained immune response against the antigen product.

An object of the present invention is to provide for the use of immunoisolating implantable devices enclosing genetically engineered antigen-producing cells to elicit immune responses against a transgene antigen in a host.

Another object of the present invention is to provide for the use of immunoisolating implantable devices enclosing genetically engineered antigen-producing cells as a vaccine.

In accordance with the present invention, there is provided the use of encapsulated genetically engineered antigen-producing cells, such as myoblasts, although one skilled in the art would understand that other antigen-producing cells may be used, encoding one or more transgene antigen, including weak antigens, as a means of providing a continuous internal source of antigen product for the purpose of eliciting a complete and strong immune response. In another embodiment of the present invention, more than one antigen may be produced and secreted by the encapsulated cells. Therefore, the present invention additionally provides a new means of vaccinating individuals against weak antigens, which otherwise could not conventionally produce a strong immune response.

In accordance with the present invention, there is provided a method for eliciting immune responses in a host, such as in mammals, and more particularly, in humans. Accordingly, selected types of recombinant antigen-producing cells, such as myoblast cells, are genetically engineered by the introduction of a DNA vector comprising a transgene encoding a specific antigen transgene. The recombinant cells may be genetic engineered in vitro by the use of various established techniques, such as transfection, electroporation (non-viral vectors), or transduction with viral vectors or any other technique capable of introducing genetic material into the cells, so as to result in recombinant cells that actively and continuously produce and/or secrete a selected antigen (the transgene product). Once engineered, the recombinant cells can be stored frozen until needed.

When needed, the cells are enclosed in immunoisolating implantable devices, such as alginate-polylysine microcapsules, or any other biocompatible non-antigenic microcapsule, following various established encapsulation protocols. A small number of encapsulated cell, for example, 10⁶ cells in a mouse, or the equivalent in a human, are implanted intraperitoneally, or via any other appropriate means, in a host where the encapsulated cells secrete antigen continuously through the microcapsules. In accordance with the teachings of the present invention, the continuous delivery of antigen stimulates an immune response in the host aimed at eliminating the source of the antigen. However, due to the encapsulation of the recombinant cells in microcapsules, the cells are protected, and the immune system can not eliminate the source of the antigen. Therefore, the encapsulated recombinant antigen-producing cells continue to provide a sustained infusion of antigen to the host, wherein the continuous delivery of antigen elicits a strong and sustained, and in one aspect, a complete, immune response, even against weakly immunogenic antigens. Furthermore, the present invention maybe used to immunize against any antigen, not only weak antigens. Nevertheless, the present invention may be particularly suitable for eliciting immune responses and immunizing against weak antigens for which conventional vaccination techniques are not generally suitable.

There is provided a method of immunizing a host, comprising administering to the host an appropriate amount of non-antigenic implantable devices enclosing genetically engineered antigen-producing cells for producing a continuous infusion of antigen to the host to elicit the activation of the immune system against the antigen. In an embodiment of the invention, the non-antigenic implantable device comprise biocompatible microcapsules. In another embodiment of the invention, the genetically engineered antigen-producing cells comprise myoblast cells, or C2C12 mouse myoblasts cells, or JPW01 mouse myoblasts cells mammalian cells or human cells. In yet another embodiment of the invention, the activation of the immune system comprises the activation of the humoral and cellular responses of the immune system against the transgene antigen. In yet another embodiment of the invention, the activation of the immune system elicits an anti-tumorigenic immune response.

The present invention also provides a method of vaccinating a host against infectious agents, comprising administering to the host an appropriate amount of a non-antigenic implantable devices enclosing genetically engineered antigen-producing cells for producing a continuous infusion of antigen to the host to elicit the activation of the humoral and cellular responses of the immune system against the antigen. In various aspects of the present invention, the host is a mammal, and in one aspect a human. The present invention can be used for wild or domesticated animals, e.g., a farm or a zoo animal.

The present invention also provides a vaccine comprising an appropriate amount of non-antigenic implantable devices enclosing genetically engineered antigen-producing cells for producing a continuous infusion of antigen to the host to elicit the activation of the immune system against the antigen.

In another embodiment, there is provided a vaccine comprising an appropriate amount of non-antigenic implantable devices enclosing genetically engineered antigen-producing myoblast cells for producing a continuous infusion of antigen to said host to elicit the activation of the humoral and cellular responses of the immune system against the antigen.

The present invention also provides a method of inducing an immune response in a host, wherein the method comprises: enclosing genetically engineered antigen-producing cells, comprising one or more transgene encoding antigen, into an immunoisolating implantable device, to provide encapsulated antigen-producing cells to a host; implanting the encapsulated antigen-producing cells in the host; production of one or more transgene antigen product in the enclosed cell; bi-directional passage of the produced antigen product through the pores of the implantable device, with preclusion of the passage of the antigen-producing cells therethrough; delivery of a continuous infusion of antigen to the host; activation of the immune system in response to the antigen.

The present invention also provides a method of vaccinating a host against cancer, comprising administering to said host an appropriate amount of a non-antigenic implantable device(s) enclosing genetically engineered antigen-producing cells for producing a continuous infusion of antigen to said host to elicit a sustained and continuous activation of the humoral and cellular responses of the immune system against said antigen; wherein said responses of the immune system are anti-tumorigenic.

The present invention also provides a method of treating a mammal suffering from cancer, said method comprising administering to said mammal an appropriate amount of a non-antigenic implantable device(s) enclosing genetically engineered antigen-producing cells for producing a continuous infusion of antigen to said mammal to elicit a sustained and continuous activation of the humoral and/or cellular responses of the immune system against said genetically engineered antigen; wherein said response(s) of the immune system are anti-tumorigenic.

### BRIEF DESCRIPTION OF THE DRAWINGS

FIG. 1 Encapsulated C2C12 myoblasts photographed under light microscopy, wherein the microcapsules in the figure have a diameter of approximately 300 μm.

FIG. 2 Detection of factor IX delivery in immunocompetent C57BL/6 mice implanted with encapsulated C2C12 myoblasts secreting human factor IX. The figure legend identifies the various lines of the plot, wherein each line represents a different clone of genetically engineered C2C12 cells that were evaluated. At various times mice were bled and plasma obtained. The concentration of hFIX in plasma was determined by an ELISA assay, as previously described (Hortelano et al., 1996). FIG. 2 shows that all clones tested, with the exception of the control, had detectable levels of hFIX that lasted for up to 21 days.

FIG. 3 Anti-hFIX antibody titre in immunocompetent C57BL/6 mice implanted with encapsulated C2C12. The presence of antibodies to hFIX in immunocompetent C57BL/6 mice implanted with encapsulated C2C12 myoblasts secreting human factor IX and shown in FIG. 2 was evaluated using an ELISA assay previously described (Hortelano et al., 1996). FIG. 3 shows an increasing titre of anti-hFIX antibodies in implanted mice starting around day 15. FIG. 3 illustrates the ratio between the optical density of a mouse on a given day (ODₓ) over the optical density of the same mouse on day 0 (ODo).

FIG. 4 Antibody titre in C57BL/6 mice implanted with encapsulated C2C12 myoblasts secreting human factor IX (hFIX). Immunocompetent C57BL/6 mice were implanted with encapsulated C2C12 myoblasts secreting human factor IX. At various times mice were bled and plasma obtained. The concentration of antibodies anti-hFIX in mouse plasma was determined by an ELISA assay, as previously described (Hortelano et al., 1996). FIG. 4 illustrates that the antibody titre increased throughout the length of the experiment (>200 days), reaching extremely high titer. Antibody titer was calculated by determining the maximum possible dilution of mouse plasma that would still yield positive results in an ELISA assay. By the end of the experiment, plasma diluted 1:500,000 would still yield positive results in the antibody assay. Therefore, these findings indicate that encapsulated C2C12 myoblasts elicit a strong humoral immune response.

FIG. 5 Cytotoxic T lymphocyte (CTL) assay in C57BL/6 mice implanted with encapsulated C2C12 myoblasts secreting human factor IX (hFIX). Mice were sacrificed at 213 days and the spleens were removed. Splenic CTL were examined following secondary in vitro stimulation. Isolated spleen cells were incubated with recombinant syngeneic recombinant cells either expressing hFIX or not expressing hFIX, and allogeneic (SVBalbC) targets at effector to target ratios of 10:1, 30:1, 100:1 in a 6 hours Cr⁵¹ release assay. Specific target lysis were calculated as follows:

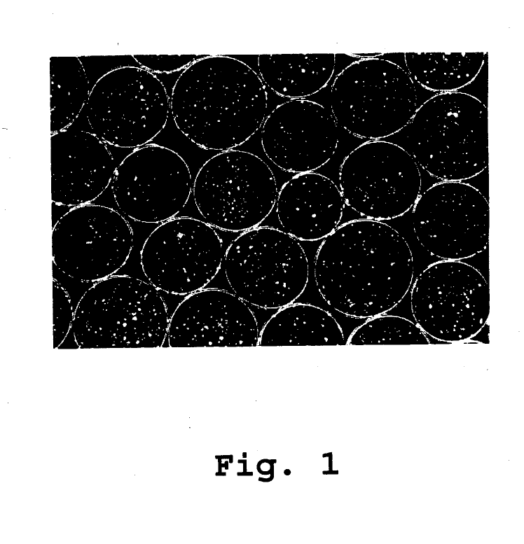

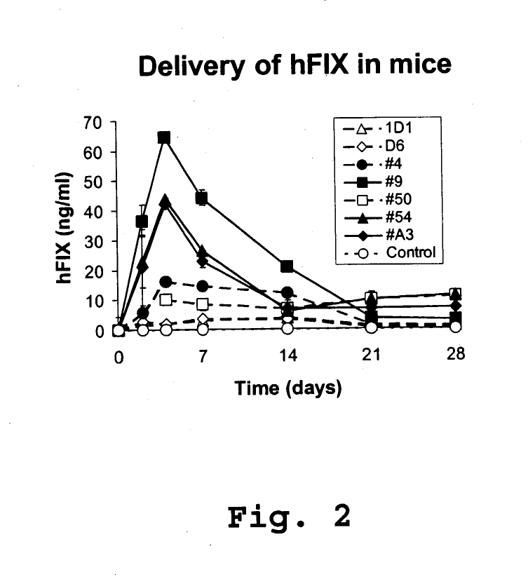

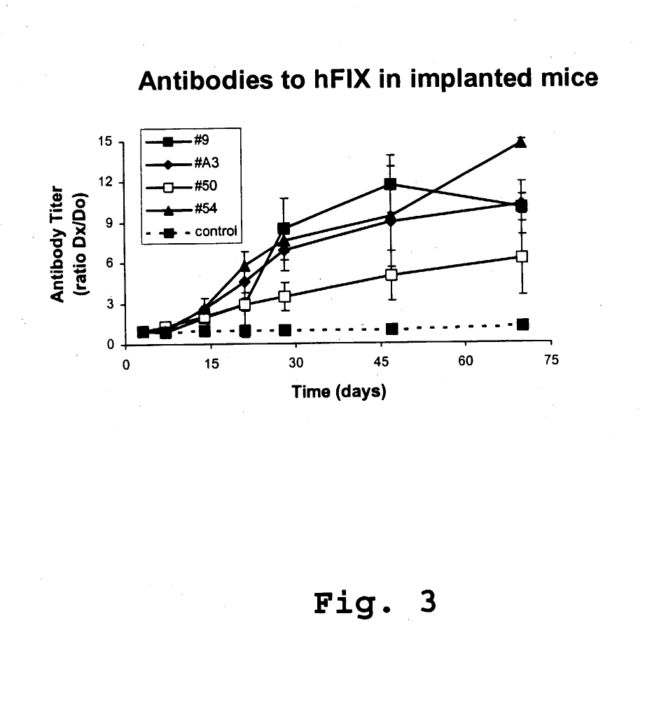

$$\frac{Experimental release - spontaneous release}{Maximum release - spontaneous release} \times 100$$

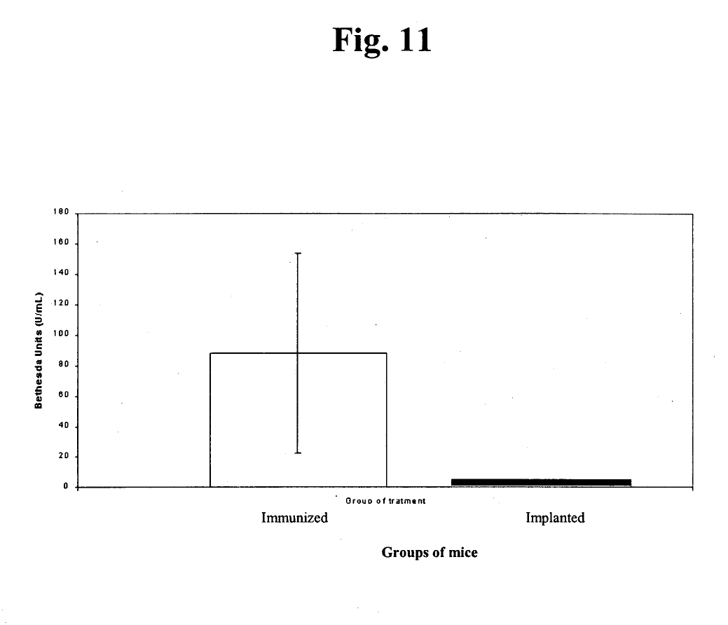

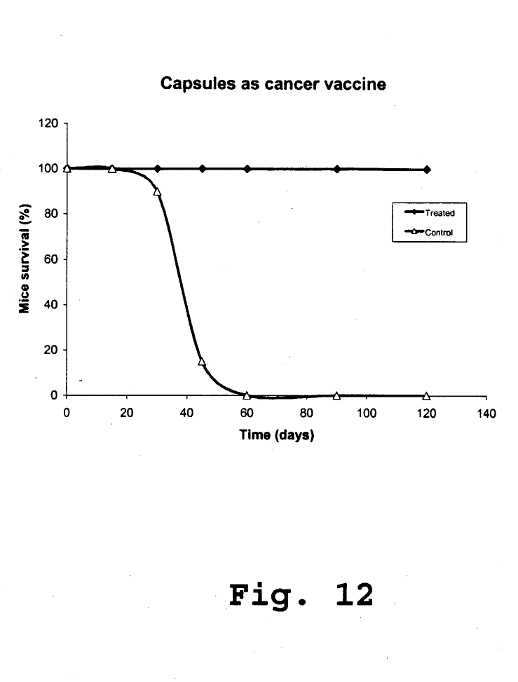

Recombinant syngeneic recombinant cells infected with adenovirus which contains the glycoprotein B gene from HSV-1 were used as a negative control for lysis by effector cells to account for non-antigen-specific lysis. FIG. 5 illustrates a strong CTL response in treated mice, indicating that encapsulated C2C12 myoblasts elicit a strong cellular immune response. Therefore, in accordance with the embodiments of the present invention, encapsulated cells elicit a strong humoral as well as a strong cellular immune response.

FIG. 6 HFIX in the plasma of immunocompetent C57BL/6 mice implanted with encapsulated C2C12 myoblasts secreting hFIX. Mice were bled at regular intervals and plasma obtained. The concentration of hFIX in mouse plasma was determined by ELISA. hFIX was detected for up to 14 days, before it became undetectable.

FIG. 7. Levels of plasma antigen by ELISA. Each line represents average of plasma hFIX in each group of treated mice with standard deviation indicated foe each time point. (♦) Implanted C57BL/6 mice (n=5), (▪) MHC I −/− C57BL/6 mice (n=5), (▴) MHC II −/− C57BL/6 mice (n=5), ( [symbol] ) CD4 −/− C57BL/6 mice (n=5), (▪) CD8 −/− mice (n=5) and (●) immunized mice (n=5).

FIG. 8. Subclasses of anti-hFIX immunoglobulins in different groups of C57BL/6 mice. Each panel shows antibody titers for Ig G subclass-specific ELISA (A: I g G total; B: Ig G1; C: Ig G2a; D: Ig G2b). Each line represents the average of each group of mice with standard deviation indicated for each time point. The assay is linear on a semilog scale. (♦) Implanted C57BL/6 mice (n=5), (▪) MHC I −/− C57BL/6 mice (n=5), (▴) MHC II −− C57BL/6 mice (n=5) ( [symbol] ) CD4 −/− C57BL/6 mice (n=5), (▪) CD8 −/− mice (n=5) and (●) immunized mice (n=5).

FIG. 9. A, Proliferation in response to stimulation with hFIX was measured by [³H]thymidine incorporation. Bars represent average of each group of mice with standard deviation indicated. ( [symbol] ) Implanted C57BL/6 mice (n=3), ( [symbol] ) immunized C57BL/6 mice (n=3). B, IFN-□ profile of the splenocytes restimulated in vitro with hFIX. Mean±SD values are indicated. (♦) Implanted C57BL/6 mice (n=3), (▪) immunized C57BL/6 mice (n=3).

FIG. 10 Production of cytotoxic T cells in cultures of splenocytes. (♦) Implanted C57BL/6 mice (n=3), (▪) immunized C57BL/6 mice (n=3), (▴) naïve C57BL/6 mice. All experimental values represent averages of three wells.

FIG. 11. Presence of antibodies that interfere with coagulation was assessed by the Bethesda assay and reported as Bethesda units. Bars represent average of each group with ±SD. ( [symbol] ) Implanted C57BL/6 mice (n=5), ( [symbol] ) immunized C57BL/6 mice (n=5).

FIG. 12. illustrates a graphic representation of survival rates of mice implanted with encapsulated JPW01/LacZ cells vs. untreated mice, when treated with an untransfected myoblast cell line.

### DETAILED DESCRIPTION OF THE EMBODIMENTS

To determine if a complete and sustained immune response may be generated against a weakly immunogenic antigen, we engineered mouse myoblasts to secrete relevant amounts of human factor IX (hFIX) and subsequently encapsulated the genetically engineered antigen-producing myoblasts cells into immunoisolating implantable devices, such as alginate-poly-L-lysine-alginate microcapsules, in accordance with one embodiment of the present invention. The encapsulated myoblasts may be implanted into patients to supply prophylactic levels of therapeutic products, such as factor IX, continuously and systemically. Alternatively, implantable devices of the present invention may be administered as an effective treatment regime against a disease and/or condition. The biocompatible non-antigenic microcapsules comprise a pore size that allows the bi-directional free flow of molecules with molecular weight of up to that of the antigen, in this case, hFIX, but not to immune cells, i.e. antigen-producing cells. Thus, this immune-isolation approach could allow for the implantation of non-autologous encapsulated genetically engineered cells into multiple recipients, making immunization both viable and economical. According to a one embodiment of the present invention long term stimulation of the immune system is elicited in accordance herewith.

The use of encapsulated genetically engineered recombinant cells can be, but are not limited to, myoblast cells, encoding one or more transgenes encoding antigen, wherein the antigen may be a weak or strong immunogenic antigen, which may or may not conventionally produce an immune response when using the techniques of the prior art, as a means of providing a continuous internal source of antigen for eliciting a full (i.e. humoral and cellular) and strong immune response. Therefore, the present invention provides a new means of vaccinating a host against antigens, which conventionally could not be immunized or vaccinated against, for example, antigens that elicit weak or no immune response. The embodiments, methods and uses of the present invention allow antigens, which would not otherwise produce a strong and sustained immune response to elicit strong and sustained immune response, and, in one aspect, elicit a strong and sustained complete immune responses. Accordingly, the present invention provides novel approach to vaccination.

The present invention additionally provides for the use of microencapsulated antigen-producing cells, genetically engineered to contain genetic material coding for one or more antigen(s), for the purpose of eliciting an immune response in a host. Although myoblast cells have been used in various experiments of the present invention, it is understood by one skilled in the art that various other appropriate antigen-producing cells may be used.

The data obtained illustrates that encapsulated myoblasts can be used as a novel method to elicit immune responses in a host have been generated using C2C12 mouse myoblasts. Although C2C12 is an established cell line, other cell types have also elicited antibodies to transgenes in the host following implantation of encapsulated cells, as discussed further hereinbelow, and for example, MDCK canine kidney epithelial cells (Garcia-Martin C, et al, Journal of Gene Medicine 4(2):215-223, 2002), or Ltk-mouse fibroblasts delivering human growth hormone (Chang P L, et al, Human Gene Therapy 4(4):433-440,1993), and β-glucuronidase (Ross C J, et al, Human Gene Therapy 11(15):2117-2127, 2000). These various cell types are established, not primary. According to a teaching of the invention, it has been shown that encapsulated mouse primary myoblasts do not elicit antibodies in implanted C57BL/6 mice. Therefore, it can be noted that that not all myoblasts would be suitable for eliciting immune responses in a host, at least not without additional genetic engineering. Accordingly, it can be noted that the more the encapsulated cell is distant in origin from the host, that is, from a different species or with a certain degree of transformation, the higher the immune response that would be expected. Nevertheless, any encapsulated cells that have been genetically modified to elicit a strong immune response by means of the processes and methods provided in the present invention may be used in accordance with the uses of the present invention.

According to an embodiment of the present invention, there are provided microencapsulated genetically engineered, antigen-producing cells, e.g., myoblast cells, encapsulated in an implantable device, or non-antigenic microcapsules, alginate-poly-L-lysine microcapsules, for example.

According to an embodiment of the present invention, the encapsulated genetically engineered antigen-producing cells may produce and secrete a single antigen product. In another embodiment of the invention, more than one antigen may be produced and secreted by the encapsulated cells. More specifically, the antigen-producing cells may comprise one or more trangene encoding different antigen products, or the microcapsules may comprise more than one type of genetically engineered antigen-producing cells. For example, the microcapsule may comprise different antigen-producing cells which comprise different trangenes. According to this aspect of the present invention, encapsulated genetically engineered antigen-producing cells may be adapted to simultaneously provide a recipient with a plurality of antigens when implanted in vivo so as to protect the recipient against a plurality of diseases and/or conditions simultaneously. Alternatively, encapsulated genetically engineered cells of the present invention may be adapted to simultaneously provide a recipient with a plurality of therapeutic products when implanted in vivo for effectively treating one or more disease(s) and/or condition(s) thereof.

In connection with the immunoisolating implantable device, which may additionally referred to herein as non-antigenic implantable device, implantable device or more simply, biocompatible microcapsule, the implantable device does not necessarily need to comprise an alginate capsules. Immune responses to transgenes have been observed in mice treated with a variety of implantable devices. Therefore, one skilled in the art would understand that a variety of implantable devices may be used, and various encapsulation procedures may be employed. Accordingly, the present invention is not limited to the use of a particular formulation of capsules, such as alginate-poly-L-lysine microcapsules, or indeed to just one particular type of implantable devices.

An aim of the present invention is to elicit immune responses against a target antigen in a host. In one aspect, an object of the present invention is to elicit a complete (humoral and cellular immunity) and sustained immune response against any transgene antigen, including both strong and weak antigens. The full activation of the immune system may be elicited in response to the continuous delivery of antigen from implanted microencapsulated cells. The processes and methods of the present invention are economically beneficial. Although the preparation of encapsulated antigen-producing cells may be a costly process, there is an advantageous cost benefit once cells have been produced, since the subsequent antigen supply is continuous, there are significant long term cost advantages. The encapsulated antigen-producing cells of the present invention may be employed as vaccines for providing prophylactic protection against infection and/or disease or alternatively, the cells of the present invention may be used in the treatment of disease and/or infection. For example, the encapsulated antigen-producing cells of the present invention may be used to elicit an immune attack against a tumor or an infectious agent, such as HIV, in vivo.

We have previously reported the use of an implantable device such as alginate microcapsules to deliver hFIX in mice having clinically relevant levels of hFIX in their plasma, suggesting the efficiency of this kind of gene therapy approach for hemophilia B. As noted above, hemophilia B is caused by a defect or deficiency in FIX, and more particularly, hFIX. Accordingly, providing hemophilia B patients with a continuous and sustained infusion of hFIX is a method of gene therapy that may treat or provide therapy for hemophilia patients.

The immune responses elicited by encapsulated cells may be different from those produced by the continuous infusion of protein or others types of gene therapy. The immune responses may differ because of the site of administration, the molecular signals between the host and the implanted cells which could generate activation of the immune system in different ways, possibly some of them unknown, doses, and the response produced against the device by itself. Uptake of the transgene product by antigen presenting cells (APC) will produce the processing and presentation of peptides of the protein by MHC-II to the T helper cells (CD4+). The T helper (Th) cells can be divided into two subclasses, Th1 and Th2. The Th2 cells help B cells, whereas Th1 cells activate macrophages. In animals models (for example, in mice), depending on various factors, such as the local concentration of cytokines, the T helper cells directs the activation of two different pathways of the immune response, Th1 or Th2. The activation of the Th2 cells results in production of IL-4 and IL-10, which stimulates B cells, resulting in the production of antibodies, principally IgG1 isotype antibodies, which is typical of immune responses against the infused proteins. The activation of Th 1 cells is characterized by the production of IL-2 and IFN, generates the proliferation of cytotoxic T lymphocytes (CTL) and B-cells secreting IgG2a, which is a classic reaction of the host to protein expressed in the context of viral infections.

FIX is a generally weak antigen, and although hFIX is a human coagulation factor, it is a weak antigen in C57BL/6 mice, and as such it is tolerated in C57BL/6 mice, since these mice generally do not respond to hFIX as being foreign, and accordingly, would not mount an immune response against hFIX. Accordingly, hFIX was an ideal candidate antigen for experiments conducted to support the teachings of the present invention. The experiments demonstrate that the methods of the present invention can elicit a strong and sustained immune response to hFIX, and can elicit a complete (humoral and cellular) immune response. Therefore, the present invention provides for a method of immunizing a host against antigens. More specifically, the present invention provides a method of immunizing a host against antigens that conventionally could not be immunized against, such as weak antigens. Moreover, there is provided a novel method of vaccination, and in particular a novel method of vaccination against weak antigens. One aspect of the present invention also provides a novel method of vaccination against or treatment of terminal diseases, such as cancer and infectious diseases, for example.

Although hFIX is a human blood coagulation factor, hFIX is weakly immunogenic, when used in experiments with mice, hFIX is not generally recognized as a foreign molecule in C57BL/6 mice, and as such an immune response is not mounted against hFIX in said mice, thereby providing an ideal antigen model for experiments leading to the developments of the present invention. The method of the present invention allows for biocompatible microcapsules containing hFIX-secreting cells engineered to deliver high levels of hFIX in vivo to be implanted in a host. Encapsulation protects the hFIX-secreting cells from both humoral and cellular immune responses of the host, thus minimizing rejection of the allogenic implanted cells.

Generally speaking, tumour antigens that are specific to cancer cells are considered to be weak antigens. The reason being the fact that after the cancer cells are considered “self” and not “foreign” in nature. Therefore, it is often difficult to elicit a strong immune response against tumour antigens. Accordingly, the present invention provides for the use of the present processes and methods to elicit an immune response, or vaccinate against any antigen, including weakly immunogenic antigens, and accordingly includes various tumour antigens.

We have shown that implantable alginate microcapsules enclosing recombinant myoblasts can deliver clinically relevant levels of hFIX in mice (Hortelano G., et al., Blood 87(12):5095-5103, 1996; Hortelano G., et al., Hum Gene Ther 10(8):1281-1288, 1999). Further, while treated nude hemophilia mice had sustained levels of FIX for at least 11 weeks (end of experiment; Hortelano G., et al., Haemophilia 7(2):207-214, 2001), FIX delivery in treated immunocompetent C57BL/6 mice was transient. This decrease in hFIX levels in vivo was concurrent to the detection of anti-hFIX antibodies. Interestingly, encapsulated cells retrieved from implanted mice still secreted hFIX in vitro at the pre-implantation rate.

More specifically, we have also shown that normal immunocompetent C57BL/6 mice, implanted intraperitoneally with 5 ml of microcapsules containing 5×10⁶ recombinant myoblast producing hFIX, all C57BL/6 mice had hFIX in their plasma, up to 65 ng/ml on day 4, decreasing to 5 ng/ml by day 21. Although on day 70, hFIX was not detected, the retrieved implanted microcapsules still secreted hFIX in vitro at the same rate as they did before pre-implantation. Thus, the decrease in hFIX levels in mice seen 21 days after implantation was not caused by a loss of microcapsules, nor the death of encapsulated cells in vivo, nor by a reduced level of hFIX secretion by the encapsulated cells. The decrease in hFIX levels was likely due to anti-hFIX antibody production. In fact, anti-hFIX antibodies were detected in all implanted mice as early as 14 days post-implantation. This pattern of increasing antibodies to the foreign hFIX coincided with a reduction in hFIX levels detectable in the plasma of all the treated immunocompetent mice.

In nude athymic C57BL/6 mice implanted with hFIX producing recombinant myoblast cells, hFIX levels were sustained for the duration of the study, namely 6 weeks. Various other experiments have shown that expression of hFIX in immunocompetent mice persist in adeno-associated viral vector transduced muscle fibers, despite the presence of neutralizing antibodies against the non-species-specific transgene product. In these other experiments, the absence of a cellular immune response, in the presence of a humoral immune response, might reflect the intrinsic properties of the vector vehicle used for protein delivery.

The experiments of the present invention provide support for the mounting of a complete (i.e. humoral and cellular) and sustained immune response to an antigen secreted from implanted encapsulated antigen-producing cells. The experimental model and results provided illustrate the induction of cellular and humoral immune responses to a weakly immunogenic antigen (namely, hFIX in C57BL/6 mice) was produced and secreted in a host in a sustained manner, by the encapsulated antigen-producing cells. More specifically, the present invention provides a process wherein encapsulated antigen-producing cells elicit strong and sustained immune responses, namely both cellular and humoral immune response; against the produced antigen, in this case, human factor IX. Therefore, the present invention additionally provides for the use of encapsulated cells as a novel delivery mechanism for antigens. According to the observations reported herein, this method has been shown may be particularly suitable for eliciting a humoral and cellular immune response against poor, or weakly immunogenic, antigens.

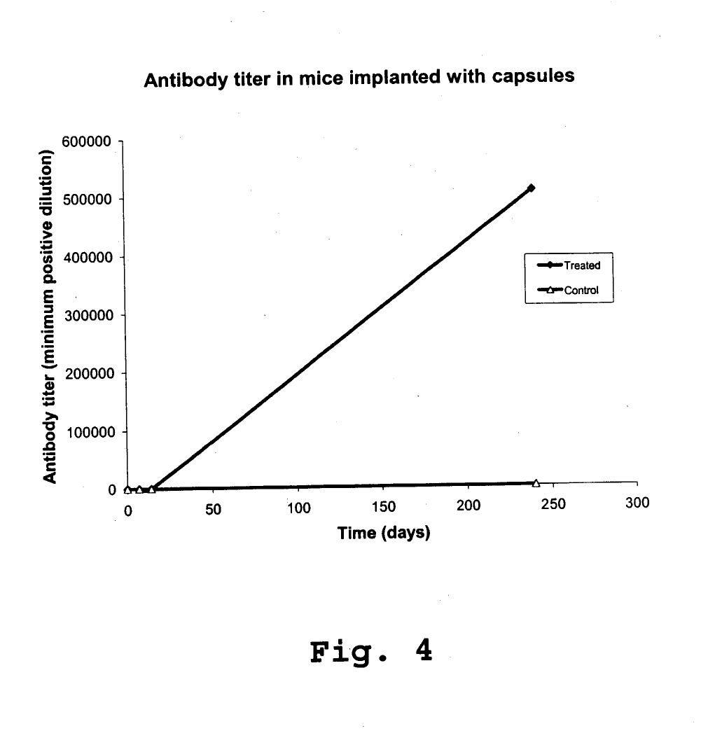

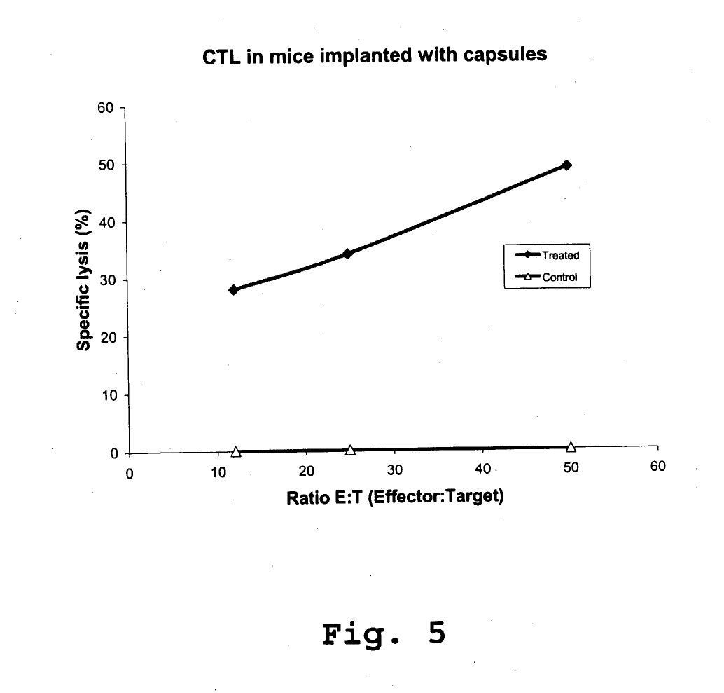

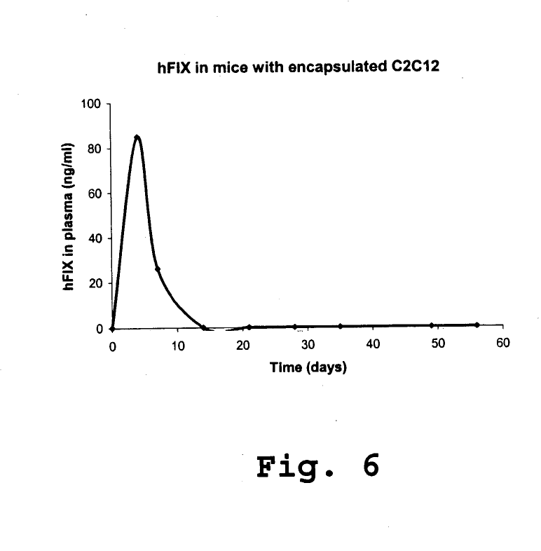

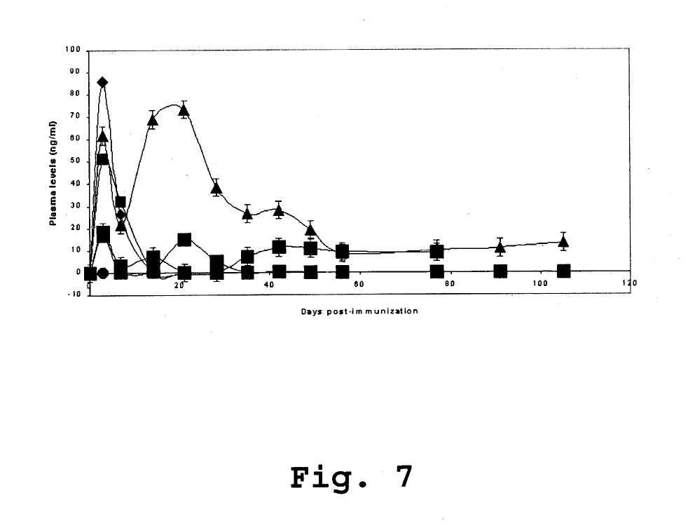

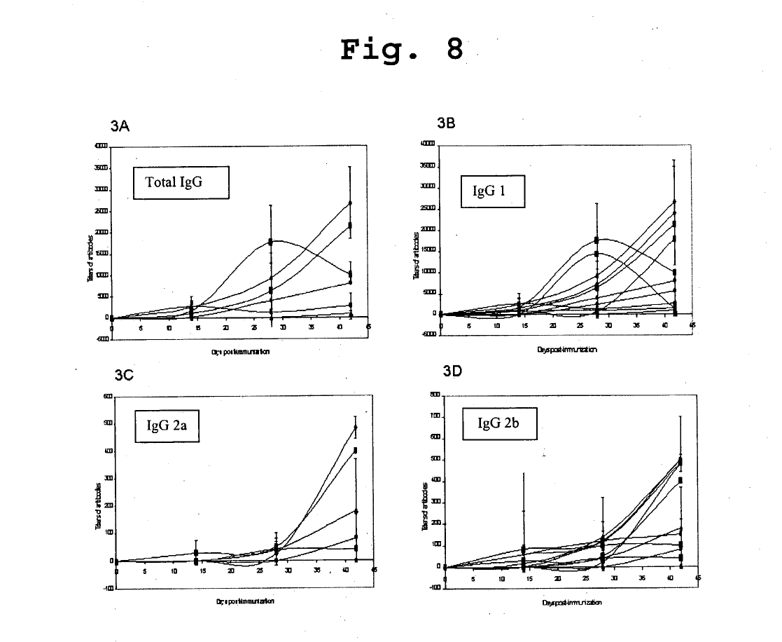

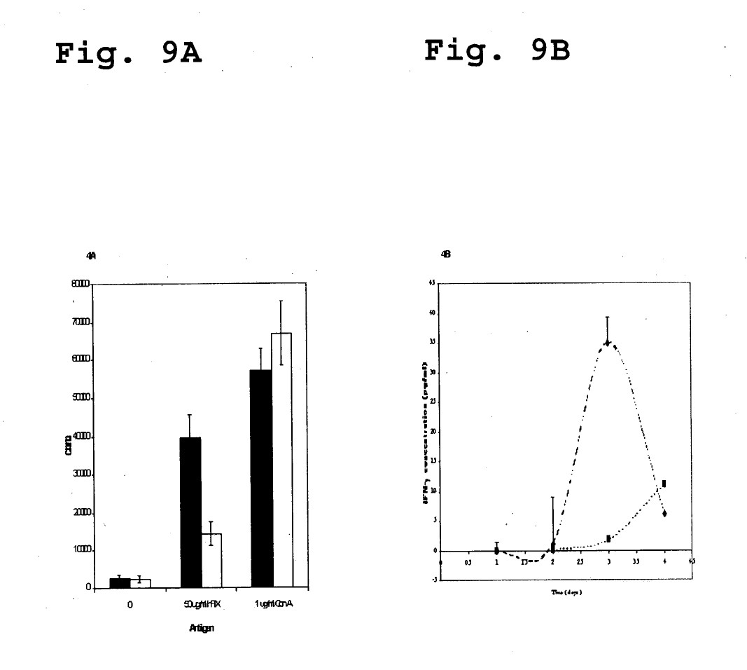

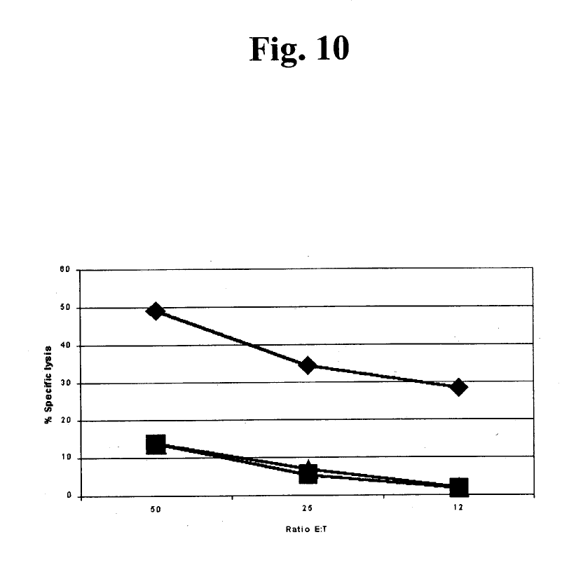

As illustrated in FIG. 4, the antibody titre in C57BL/6 mice implanted with encapsulated C2C12 myoblasts secreting human factor IX (hFIX) clearly shows a strong and sustained immune response to hFIX (humoral immune response). In addition, as illustrated in FIG. 5, cytotoxic T lymphocyte (CTL) assay in C57BL/6 mice implanted with encapsulated C2C12 myoblasts secreting hFIX, a high CTL response indicates a strong cellular immune response against hFIX in treated mice. This immune response is further supported by the secretion of interferon γ by splenocytes from treated mice 63 days after microcapsule implantation. Therefore, both arms of the immune system are activated. It is important to consider that C57BL/6 mice are generally considered not to elicit an immune response against hFIX.

The present invention will be more readily understood by referring to the following examples which are given to illustrate the invention rather than to limit its scope.

### EXAMPLE I

### hFIX Secretion by Encapsulated Cells

Materials and Method

Tissue Culture Conditions

Mouse C2C12 recombinant myoblast secreting hFIX was described elsewhere (Hortelano G, et al., Hum Gene Ther 10(8): 1281-1288, 1999), cultured under standard conditions in Dulbecco's modified essential medium (DMEM) containing glucose (4.5 g/liter), 1% penicillin/streptomycin (GIBCO, Burlington, Canada), and 10% fetal bovine serum (GIBCO). Myoblast in microcapsules were cultured in vitro in 600 ml flask (Nalge Nunc), with 1 ml of capsules per 10 ml of medium under regular tissue culture conditions.

Enclosure of Recombinant Myoblast

Encapsulation of recombinant myoblast was performed as described earlier (Hortelano G, et al., Blood 87(12): 5095-5103, 1996) with some minor modifications. A suspension of cells was mixed with 1.5% potassium alginate (Kelmar; Kelco, Chicago, Ill.) in a syringe and extruded through a 27-gauge needle with a syringe pump (39.3 ml/hr). An air jet concentric to the needle created fine droplets of cell-alginate mixture, which were collected in a 1.1% CaCl₂ solution. Upon contact, the droplets gelled. The obtained beads were washed in a number of solutions, as previously described (Chang, P. L., et al, Biotechnology and Bioengineering, 43:925-933, 1994). The outer alginate layer was chemically cross-linked with poli-L-lysine hydrobromide (PLL; Sigma, St. Louis, Mo.) with a molecular weight ranging from 15,000-30,000, for 6 min, and then with another layer of alginate. Finally, the remaining free alginate core was dissolved with sodium citrate for 6 min, to yield microcapsules with an alginatePLL-alginate membrane containing cells. After the microcapsules are made, they are kept in regular tissue culture medium until implanted into animals. All the steps should be performed under sterile conditions.

The hFIX secretion by encapsulated cells before implantation and after retrieval of implanted microcapsules was determined by culturing them and sampling media aliquots at time intervals. The number of microcapsules per 100 ul were also quantified, and then, released the encapsulated cells by gentle pressure, and a sample of 40 μl on a hematocitometer, to count the number of cells for implantation. Also, a sample of microcapsules was placed on a slide with trypan blue and, after a gentle pressure was applied with a coverslip, the microcapsules released the enclosed cells, to determine the cell viability.

Recombinant Cells are Expressing Two Transgenes

Vector pLNMβIXIL has been described elsewhere (Hortelano et al., 1999). The expression vector contains the cDNA of hFIX under the control of the β-actin promoter, and also includes the muscle-specific MCK enhancer. In addition, the vector contains a second transgene, the neo^{r} cDNA that confers resistance to the antibiotic neomycin to the cells that express it. Mouse C2C12 myoblasts were transfected with pLNMβIXIL by the calcium phosphate precipitation method. Individual recombinant clones that express the neo^{r} cDNA were identified after selection of the cells in G418 (400 μg/ml), by ELISA assay, as described (Hortelano et al., 1996). Further, clones were screened for secretion of hFIX. Therefore, selected clones were expressing two transgenes, hFIX and neo. All vector construction and modifications were performed following standard molecular biology techniques.

Animal Experiments

Five normal C57BL/6 mice (Charles River Breeding Laboratories), five CD4− (Taconic), five CD8− (Courtesy Dr. Wan, Center of gene therapy, McMaster University), five MHC I knockout (Jackson Lab), and five MHC II knockout (Jackson lab) were anesthetized by inhalation of isofluorane, and implanted intraperitoneally with 3 ml of microcapsules containing 3×10⁶ viable myoblast (clone 18), the first 3 groups, and 4×10⁶ the latest 2 groups, with a 16-gauge catheter. All mice have been bled retroorbitally on a weekly schedule up to 8 weeks post implantation, and then biweekly until the end of the experiment, using heparinized hematocapillary tubes. Plasma was obtained and stored at −20° C. All experiments and techniques were performed according to Canadian Animal Ethics guidelines.

Implantation of Microcapsules in Mice

Normal male Normal male C57BL/6 mice (Charles River, Canada) were implanted with microcapsules enclosing factor IX-secreting myoblasts. Immediately before implantation, the microcapsules were washed 5-6 times in Hanks solution (Gibco, Burlington, Canada), or other physiological solution (Ringer's solution). The animals were anesthetized using a small-animals anesthetic machine (Med-Vet, Toronto, Ontario) providing a controlled amount of isofluorane (Anaquest, Mississauga, Ontario), oxygen and nitrous oxide. The implantation procedure was done using I.V. Catheter (Angiocath, 16 G) introduced into the peritoneal. cavity. The whole procedure took about 5 min and the animals were soon freely mobile in their cages. Animals were typically implanted with up to 5 ml of microcapsules (90% packed capsules volume in Hanks). At the end of the procedure animals have a bloated abdomen, but this condition disappeared in 24 h, after most of the fluid was eliminated. All experiments and techniques were performed according to Canadian Animal Ethics guidelines.

Fetal Myoblasts do not Elicit Immune Response in C57BL/6 Mice

G8 mouse myoblasts (ATCC No. CRL-1456) were obtained from muscle tissue of a murine fetus. Fetal G8 myoblasts were genetically engineered to express hFIX. Immunocompetent C57BL/6 mice were implanted with G8 myoblasts secreting hFIX that were enclosed in alginate-poly-L-lysine microcapsules. Mice were bled at regular times, and plasma obtained. Levels of hFIX antigen and anti-hFIX antibodies in plasma were determined by ELISA assays, as previously described (Hortelano et al., 1996). In contrast with our findings using C2C12 myoblasts, mice treated with encapsulated G8 myoblasts had sustained levels of hFIX for at least 60 days, however, these treated mice did not elicit antibodies to hFIX. Accordingly, these findings indicate that not all encapsulated cells elicit an immune response, and that the selection of the encapsulation device, in addition to the antigen produced is important and must be carefully considered.

Assays for Human Factor IX Antigen and Anti-hFIX

The plasma samples were used to detect hFIX and anti-hFIX antibodies by enzyme-linked immunosorbent assay (ELISA). For the detection of hFIX, microtiter plates (GIBCO) were coated with 1:100 sheep anti-hFIX in 100 ul of 0.1M Na2CO3 pH 9.6 for 2 hours a 37° C. After blocking with 5% skim milk powder at 4° C. for 18 hours, each well was incubated with 100 ul of normal mouse plasma (1:5 dilution in 5% blotto) at 37° C. for 2 hours. Then, the wells were incubated with 100 ul of rabbit anti-hFIX (Stago) diluted 1:500 in 5% skim milk powder, at 37° C. for 1 hour, followed by goat anti-rabbit Ig G-AP conjugated (GIBCO) diluted 1:1000 in 5% skim milk powder at 37° C. for 1 hour. The substrate para-nitrophenylphosphate (Sigma) at concentration of 1.5 mg/ml was added, incubated for several minutes and read at OD 405 nm in ELISA reader. Pooled human plasma (containing 5 μg of factor IX/ml) was used as standard for hFIX.

Antibodies against hFIX were detected by subclass-specific ELISA. Briefly, microtiter plates were coated with 5 μg/ml of plasma derived human FIX protein (Courtesy Dr. Fredrick A. Ofosu) for 18 hours at 4° C., then, the wells were incubated with 150 μl of solution of 1% Bovine Serum Albumin (BSA) in Phosphate Saline Buffer (PBS) for 18 hours at room temperature, and 50 μl/well of mouse plasma samples were applied at dilutions of 1:25, 1:50, 1:100, 1:200, 1:400 and 1:500 for detection of IgG 2a and IgG 2b; and dilutions of 1:500, 1:1000, 1:2000, 1:4000, 1:8000, 1:16000 and 1:32000 for detection of IgG total and IgG 1 in a solution of 0.3% of BSA in PBS, and incubated for 18 hours at 4° C. IgG total anti-hFIX antibody was detected by rabbit anti-mouse IgG-AP conjugated (GIBCO) (in dilution 1:1000) and developed with 50 μl of para-nitrophenylphosphate as mentioned above. Other antibody isotypes were incubated with 50 μl of Streptavidin in dilution 1:1000 for 1 hour at room temperature. Antibodies levels were measured by OD reading at 405 nm follow incubation with 50 μl of para-nitrophenylphosphate. Antibody titers were determined by standard curves for wells coated with serially diluted murine IgG proteins (IgG1, IgG2a, IgG2b from Sigma). The time of developing was determined by checking two dilutions from the strongest dilution of the serial standard, which should not be too close with respect to colour intensity. In order to determine the minimum detectable dose, two standard deviations were added to the mean optical density value of the zero standard. The assay is linear on a semilog scale.

Proliferation Assay

Three normal mice were sacrificed on day 63, and the spleens were obtained for use in this assay. Spleen cells were prepared by teasing the tissue through a stainless steel grid. The splenocytes were plated at a density of 5×10⁵/well and cultured in 96 well plates for 5 days in HL-1 medium (Bio-Whitalkker) supplemented with 1 U/ml penicillin, 100 μg/ml streptomycin (both from GIBCO) and 1 μg/ml gentamicin (Sigma), with the following concentrations of human factor IX: 0, 1 μg/ml, 5 μg/ml, 10 μg/ml and 20 μg/ml. The cells were incubated in triplicate wells, and 1 μCi of [³H] thymidine was added to wells during the last 18 hours of culture. The plates were then harvested and counts per minute were determined by means of liquid scintillation counting. Cell-free supernatants were harvested each 24 hours until day 3 and analyzed for the presence of IFN-γ by ELISA. As control, we used splenocytes stimulated with Concanavalin A for 3 days at concentration of 5 μg/ml.

IFN-γ ELISA

The IFN-γ in the supernatant fluid of restimulated T-cell clones was quantified by ELISA. Wells of a 96 well plate (Corning) were coated overnight at 4° C. with 100 μl of anti-IFN-γ antibody 2 μg/ml (pharmigen) diluted in carbonate buffer. Plates were washed with PBS-0.1% Tween-20, and non-specific binding was blocked with PBS-10% fetal bovine serum (FBS, GIBCO) for 2 hours at room temperature. Samples and standards from 6 ng/ml to 9.4 pg/ml) at 75 μl per well, and incubated 12 hours at 4° C. The samples and the standard were run by duplicate. Plates were washed 4 times with PBS/Tween and biotinylated anti-IFN-γ (1 μg/ml) was added to each well. The plates were incubated at room temperature for 2 hours, then the plates were washed 6 times, a 1:1000 dilution of AP-conjugated extraavidin in PBS-10% FBS was added, and incubated at room temperature for 2 hours. Plates were washed 8 times and developed with para-nitrophenylphosphate. Plates were read at OD 405 nm. The IFN-γ was quantified by comparison with a standard curve of purified IFN-γ (Quantikine R & D systems) captured and detected.

CTL Assays

All mice were sacrificed and the spleens were removed. As illustrated in FIG. 5, splenic CTL (cytotoxic T lymphocytes) were examined following secondary in vitro stimulation as follows; isolated spleen cells were incubated for 6 days with gamma-irradiated (5,000 rad) recombinant syngeneic cells expressing hFIX, at an effector/stimulator ratio of 1:166 in HL-1 medium with penicillin, streptomycin and gentamicin. After stimulation splenic effector cells were incubated with syngeneic cells either expressing hFIX or not and allogeneic (SVBalbC) targets at effector to target ratios of 10:1, 30:1, 100:1 in a 6 hours Cr⁵¹ release assay. Specific target lysis will be calculated as follows:

$$\frac{Experimental release - spontaneous release}{Maximum release - spontaneous release} \times 100$$

All experimental values represent averages of three wells; maximum are target cells incubated with 10% SDS, and spontaneous are target cells incubated with medium alone, and are also averaged from three wells. Cells MC 57 infected with adenovirus which contains the glycoprotein B gene from HSV-1 will be used as a negative control for lysis by effector cells to account for nonantigen-specific lysis. As noted above in FIG. 5, a strong CTL response is observed in treated mice, indicating that encapsulated C2C12 myoblasts elicit a strong cellular immune response. Therefore, in accordance with the embodiments of the present invention, encapsulated cells elicit a strong humoral as well as a strong cellular immune response.

Results

Delivery of hFIX in Immunocompetent Mice

The group of normal mice immunocompetent C57BL/6 had hFIX in their plasma, with peak of 85 ng/ml detected by ELISA in day 3, as illustrated in FIG. 6 (which relates to FIG. 4 and FIG. 5). In previous experiments we have shown that administration of systemic hFIX following implantation in these animals is prevented by formation of neutralizing antibodies. Consistent with this finding, all normal mice implanted with encapsulated C2C12 producing hFIX did not have detectable levels of this protein in plasma samples by ELISA at day 14. On day 63, three mice of this group were sacrificed, and the implanted microcapsules were retrieved so as to characterize their viability and secretion of hFIX in vitro. The viability of encapsulated myoblast was 64% after the implantation, a little lower than before the implantation which was 75%; the levels of secretion of hFIX were similar before the implantation to the levels after retrieval of microcapsules, which were 325 ng/ml/10⁶ cells.

IgG Subclasses ELISA

Similar to previous experiments, hFIX delivery in mice is antigenic. At day 14 growing levels of antibodies in normal mice were detected, up to day 42, where the levels remained constant. So, the decrease in hFIX levels was likely due to anti-hFIX antibodies production. The delivery of hFIX in mice resulted in the induction of antibodies of the T helper cell-dependent isotype IgG1 principally. At day 28, an increase in IgG2a was observed in all implanted mice. There is correlation with the production of IgG2a and IgG2b and decrease of plasma levels of hFIX.

IFN-γ Assay

IFN-γ-specific ELISA were performed in order to address differences in T helper subsets activated by hFIX delivered by encapsulated recombinant myoblast, because this cytokine represent CD4+ cells in the context of Th1 response. Lymphocytes from normal mice secreted IFN-γ after stimulation with hFIX antigen indicating activation of Th1 response. Therefore, part of the immune response against hFIX after implantation is due to the activation of the Th1 helper way.

Discussion

Previous findings illustrating that implantable alginate microcapsules enclosing recombinant myoblasts can deliver clinically relevant levels of hFIX in mice (Hortelano G., et al, 1999, Human Gene Therapy). It is well known, as noted above, that hemophilia B is an X-linked bleeding disorder caused by a defect or deficiency in blood coagulation factor IX (FIX). Gene therapy may be an alternative to current therapies based on the regular infusion of plasma-derived or recombinant products. The findings of Hortelano et al (1999) suggest the potential use of this approach of gene therapy for hemophilia B. In fact, it was also been shown that nude mice treated with such gene therapy had sustained levels of FIX for at least 11 weeks (Hortelano G., et al 2001, Hemophilia), while FIX delivery in treated immunocompetent C57BL/6 mice had transient levels of FIX. This in vivo decrease in hFIX levels was concurrent with the detection of anti-hFIX antibodies. Interestingly, encapsulated cells retrieved from implanted mice still secreted hFIX in vitro at the pre-implantation rate.

The present study was designed to evaluate the immune responses to hFIX in mice implanted with microcapsules. Immunocompetent C57BL/6 mice, were implanted intraperitoneally with microcapsules containing recombinant myoblasts secreting hFIX. The levels of hFIX in the plasmas of immunocompetent C57BL/6 mice were transient, and become undetectable by day 14. At this time, the production of anti-hFIX antibodies, IgG2a and IgG2b isotypes, were detected. Subsequently, in these mice, sustained levels of hFIX were continued to be observed at low concentration until the end of the experiment, namely day 213. These findings and observations indicate that the immune response to hFIX was not exclusively mediated by a CD4 or MHC II dependent mechanism. Moreover, different intensities of T-cell mediated immune responses to hFIX were observed between the experimental mice.

Lymphocytes from immunocompetent C57BL/6 mice secreted IFN-γ after stimulation with hFIX antigen, thereby indicating the activation of a Th1 response. This accordingly indicates that part of the immune response against hFIX after implantation was due to the activation of a Th1 helper response.

The results of the present experiment confirm that the expression of hFIX by recombinant encapsulated myoblasts within microcapsules was sustained in vivo. Furthermore, the immune response to hFIX may be mediated through a CD4-independent mechanism. The present experiments indicate that the duration of hFIX expression may affect the induction of the immune responses. And more particularly, the present model illustrates that the activation of both humoral and cellular immune responses in a strong and sustained manner.

### EXAMPLE II

### Use of Continuous Antigenic Stimulation for Inmmunization

Protection against chronic infectious and neoplasic diseases is mediated through an efficient immune response. In this study we describe a novel method of immunization, herein referred to as a continuous antigenic stimulation system (CASS) based on the implantation of encapsulated cells constitutively producing an antigen. Plasma antigen was persistent in MHC II−/− and CD4−/− mice, but was only detectable in implanted C57BL/6 mice for up to seven days, as circulating antibodies became detectable. In implanted C57BL/6 mice, the humoral response was at least twice that of mice immunized with freund's adjuvant. The cellular response in C57BL/6 mice implanted with microcapsules resulted in a strong CTL activity persisting for at least 213 days after implantation. In contrast, no CTL activity was present in immunized mice by day 28 post-treatment. Additionally, activation of the immune system via a naturally fold antigen produced by continuous antigenic stimulation, as herein described, avoids cross-reactivity. This aspect of the present invention may have applications in the design of novel vaccines against life-threatening diseases.

Encapsulation of xeno or allogeneic cells might serve as an ongoing immunogen expression system. For example, we have previously implanted mice with encapsulated recombinant myoblasts secreting human factor IX (hFIX), as an ongoing protein replacement system for treatment of hemophilia B. The capsules serve to protect recombinant cells from immune responses mounted by the host. In these studies, we noted that implanted mice developed Ig G specific antibodies for hFIX, yet the encapsulated cells remain viable for 213 days.

In this study, we investigated the cell encapsulation method as a continuous antigenic stimulation system (CASS) able to enhance the priming and persistence of a specific immune response by a constant supply of antigen that enables progressive maturation to a long-term immunological memory. The objective of this study was to compare humoral and cellular immune responses elicited by the present invention, with those obtained with a conventional immunization methodology.

Materials and Methods

Encapsulation of C2C12 Myoblast

Mouse C2C12 recombinant myoblasts secreting hFIX were encapsulated as described hereinabove. Viability of the cells in retrieved microcapsules was evaluated by trypan blue exclusion (Chang P. L. et al. Trends Biotechnol. 1999 Febuary; 17(2):78-83).

Animal Experiments

C57Bl/6 mice (Charles River, Montreal, Quebec, Canada), knockout C57Bl/6 MHC II−/− and CD4−/− mice from Taconic (Germantown, NY), and knockout C57Bl/6 MHC I−/− and CD8−/− mice from McMaster University, were 6-8 wk of age when used. Mice were housed in a pathogen-and viral Ab-free facility at McMaster University. Microcapsule implantation (3×10⁶ myoblasts per mouse) was performed as described in Hortelano G. et al. Blood. Jun. 15, 1996; 87(12):5095-103, which is herein incorporated by reference.

For immunization, mice were injected s.c. in the lower limb with 500 ng of human recombinant FIX (Benefix, Genetics Institute, Cambridge, Mass.), mixed with Freund's adjuvant (GIBCO, Grand Island, N.Y.) as described in Romball C. G. et al. Eur J Immunol. 1984 October; 14(10):887-93. Blood samples were obtained from retro-orbital bleeding at regular times. All experiments were conducted in accordance with the Canadian Animal Ethics Guideline.

Assays for Human Factor IX and Anti-hFIX

Plasma hFIX (Affinity Biologicals, Ancaster, ON) and total IgG anti-hFIX antibodies (Promega, Madison, Wis.) were detected by ELISA. Also, antibodies against hFIX were detected by subclass-specific modified ELISA (Hortelano G. et al., Blood 1996). The IgG1and IgG2a isotypes of antibodies were developed with Streptavidin-AP (Sigma, St. Louis, Mo.) conjugated in dilution 1/1,000 for 1 hour at room temperature. Antibodies titers were determined by standards curves for wells coated with serially diluted murine IgG proteins, IgG1, IgG2a, IgG2b (Sigma, St. Louis, Mo.).

In Vitro Proliferation and IFN-γ Profile

Spleens from mice (n=3) sacrificed on day 28 (immunized), on day 63 (implanted) and naïve mice were obtained. Groups of cells were stimulated with Con A as a positive control. To analyze proliferative responses, 500,000 splenocytes were stimulated with (20 μg/ml) or without hFIX in 0.2 ml of HL-1 media (Bio-Whittaker, Walkersville, Md.). After 72 hours cultures were pulsed for 6 h with 1μCi [³H]thymidine (New England Nuclear, Boston, Mass.), and incorporated radioactivity was measured in a scintillation counter (LBK Pharmacia, Piscataway, N.J.) as described earlier (Gallichan W. S. et al. J Infect Dis. 1998 May;177(5):1155-61). To determine cytokine production, 5×10⁵ splenocytes were cultured in 0.2 ml of medium. Supernatants were collected after 0, 24, 48, and 72 h, and samples were run by triplicate. Levels of IFN-γ were assayed by ELISA as previously described (Quantikine, R y D, Minneapolis, Minn.) (Wenner C. A. et al. J Immunol. Feb. 15, 1996; 156(4):1442-7).

CTL Assays

Mice from the implanted group were sacrificed at day 213 and the spleens processed for a CTL assay as previously described(Gallichan W. S. et al. J Infect Dis. 1998 May;177(5):1155-61). In addition, spleens from three immunized mice that were sacrificed at day 28 after the last boosting and three naïve mice were also processed. The splenocytes were cultured in triplicate and stimulated with irradiated MC-57hFIX (syngeneic target), or SvBalb-hFIX (allogeneic target), MC-57 (syngeneic control), or MC-57glycoprotein B (syngeneic control expressing glycoprotein B from HSV-1 as an irrelevant antigen). Cytotoxic activity was evaluated at day 4. The maximum quantity was estimated by target lysis with 10% SDS, and the spontaneous denomination was target cells incubated with media alone. Briefly, splenic effector cells were incubated with the target cells at an efector/target ratio of 1:50, 1:25, and 1:12 in RPMI-10% FBS medium with antibiotics for a 6 hours Cr release assay. Specific target lysis were calculated as follows:

$$\frac{Experimental release - spontaneous release}{Maximum release - spontaneous release} \times 100$$

Modified Bethesda Assay for hFIX Inhibitors

The presence of neutralizing antibodies (inhibitors) was evaluated as described before (Kung S. H. et al. Blood. Feb. 1, 1998;91(3):784-9). Plasma samples on day 49 from implanted and immunized mice (n=5) were analyzed in a clotting aPTT assay for the presence of neutralizing antibodies. Pooled normal plasma incubated in dilution buffer was used as a control.

Results

Antigen Bioavailability

The dose and persistence of antigen following immunization determine antigen bioavailability in vivo. Our aim was to assess whether the long term effect of continuous antigen sustainability is reflected in a prolonged and effective immune response. A group of immunocompetent C57BL/6 mice and knockout mice were implanted with encapsulated myoblasts (CASS), all of which had detectable hFIX in plasma (FIG. 7). In contrast, classically immunized C57Bl/6 mice had undetectable free plasma antigen (FIG. 7). We previously reported that sustained delivery of hFIX following implantation in mice is prevented by development of antibodies against the transgene (Hortelano G. et al. Blood. Jun. 15, 1996;87(12):5095-103.). In agreement with our previous findings, none of the implanted mice had detectable antigen in plasma by day 14. All knockout mice had detectable levels of circulating hFIX (FIG. 7). Both MHC I−/− and CD8−/− groups of mice showed transient levels of hFIX, in a pattern similar to that of immunocompetent C57BL/6 mice, although free antigen persisted in CD8−/− mice for at least a month after the implantation (FIG. 7). In contrast, MHC II−/− and CD4−/− mice had detectable circulating free antigen until the termination of the experiment (day 213, FIG. 7).

At least 50% of the implanted microcapsules were retrieved from animals at day 213 post-treatment (end of the experiment). The viability of retrieved encapsulated myoblast was 69%, comparable to 75% prior to implantation. Retrieved microcapsules were cultured in vitro under regular tissue culture conditions, and the secretion of hFIX determined. Human FIX secretion before (308.8 ng/ml/10⁶ cells), and after implantation (243.4 ng/ml/10⁶ cells) was comparable, in agreement with our previous findings. Thus, we concluded that the decrease in the detection of plasma hFIX by day 14 was not due to the loss of microcapsules, death of the cells or reduced level of secretion by the cells.

Ig G Isotypes

The induced hFIX-specific antibody response in implanted (CASS) and immunized mice was compared. The presence of total IgG antibodies, as well as IgG1, IgG2a, and IgG2b isotypes against recombinant hFIX in serum was evaluated in all mice. In agreement with our published findings⁸, by day 14, detectable levels of Ig G antibodies were observed in all mice (FIG. 8), which could explain the rapid decline of circulating antigen seen in the implanted group (FIG. 8). The levels of IgG antibody induced by CASS were 3-4 times higher than in immunized mice. Interestingly, the antibody titers in implanted mice continued to increase throughout the whole experiment.

The immune response against hFIX elicited antibodies dependant of the T-helper system, principally manifested by an increase of IgG1 isotype in both implanted and immunized groups. The titers of total IgG and IgG1 were similar in all groups, indicating that most of the immunoglobulin G produced was IgG1. However, implanted mice showed the highest levels of different antibody isotypes. The disappearance of free circulating hFIX coincides with the emergence of IgG2b antibodies in implanted mice (FIG. 8). The pattern of the humoral response and the clearance of the hFIX observed in MHC I−/− and CD8−/− mice were similar to those of the implanted immunocompetent mice, although the knockout mice had a lower antibody titer.

Taken together, our findings indicate that the route of antigen presentation of hFIX by microcapsules is through MHC II class molecules and CD4+ cells. This route of antigen presentation is consistent with previous reports for this antigen, although the immune response generated by CASS seems to be stronger and more stable than those described before.

Antigen-Specific Splenocytes Proliferation and IFN-γ Assay

Splenocyte proliferative responses were also analyzed in the treated mice. Cells not stimulated with antigen, or cells from naive mice did not proliferate. The proliferative response was twofold higher in the implanted group than in the immunized group (FIG. 9). This result indicates that the immune stimulation provided in vivo by the encapsulated myoblasts was sustained.

IFN-γ was measured in order to address differences between hFIX-activated T helper subsets from mice treated with CASS as opposed to those from immunized mice. Stimulated lymphocytes from both immunized and implanted groups of mice secreted IFN-γ, indicating activation of Th1 response, although the secretion of this cytokine was lower in the immunized group (FIG. 9). In contrast, splenocytes from naive mice or cells not stimulated with antigen secreted undetectable levels of IFN-γ. The levels of IFN-γ found in the various splenocyte cultures agreed well with their ability to proliferate.

Antigen Cytotoxicity

hFIX-specific CTL were present in mice implanted with encapsulated myoblasts for at least 213 days, causing lysis of up 30-60% of target cells (FIG. 10). In contrast, minimal CTL activity, similar to the level of naive animals was found in the immunized group. Sustained delivery of hFIX by CASS resulted in highly efficacious CTL priming in treated mice.

Cross-Reactivity and Auto-Immune Disease

We used encapsulated myoblasts to investigate the consequences of continuous antigen stimulation in the function of the immune system. FIX can be used in cross-reactivity studies given the high homology between the catalytic sites of human and murine FIX. The inhibitory activity against hFIX of the immunized group was evident with a steady increase of neutralizing antibodies (FIG. 11). In contrast, the plasma of implanted animals did not show inhibitory activity at all (FIG. 11). These results were consistent in all the animals participating in the study, contrary to those results shown in previous studies.

Discussion

Antigen dosing and persistence are important factors that affect immunization. This study describes encapsulated recombinant myoblasts as a novel approach to prime the immune response via constant antigen stimulation, a strategy with potential application in the prevention and/or treatment of chronic diseases such as cancer or HIV, for example. Using hFIX as a suitable antigen model, we present data indicating that CASS is clearly more efficient in eliciting a strong and sustained cellular immune response than a classical scheme of immunization. The prolonged production of antibodies in implanted mice is consistent with persistent production of the antigen by the implantable device, suggesting that microcapsules can be used in single step immunization. The system of the present invention can accomplish priming with an effective single-dose immunization procedure, a frequent limiting factor for vaccine efficacy.

Our studies show clearly that the system of the present invention induces potent immunity to the transgene product. Furthermore, we were not able to find previous studies that were successful in the development of acquired hemophilia B in mice mediated by neutralizing antibodies or strong CTL responses induced by immunization. The kinetics of immune activation following the implantation of the microcapsules indicates that protein expression in vivo precedes and drives immune activation. The humoral immune response lagged behind protein detection in the serum by several days. This is not surprising, since is not unreasonable to expect that some protein concentration is needed to surpass the activation threshold of B cells and develop a functional Th cell response. Splenocyte proliferation occurred in both groups (implanted and immunized) following antigen administration, but the response was far greater in the implanted group. The persistence of free hFIX in the plasma of MHC II−/− and CD4−/− mice, and the activation of the immune response in the C57Bl/6 group of mice are certain to demonstrate the route of presentation of this particular secreted molecule, pattern consistent with the immune response against an infused protein ²⁸. Clearly, activation of the immune system took place in implanted as well as immunized mice. Absence of activation of CD8+ cells against hFIX in the immunized group may reflect inability to efficiently display hFIX peptides associated with the MHC class I determinants, or inability to provide an activation signal to APCs.

One of the pivotal obstacles that has hampered, and still hampers the development of effective vaccines is the requirement for efficient induction of Th1 immunity, which in general has been difficult to achieve by use of traditional vaccines. Our findings demonstrate that implantation of encapsulated recombinant myoblast resulted in activation of hFIX-specific CTLs in C57BL/6 immunocompetent mice, while a classical scheme of immunization did not. Various cytokines secreted by activated T helper cells have been shown to induce B cells to class-switch to a particular isotype. IFN-γ is the main switch factor regulating IgG2a switching, and we observed high levels of secretion of this cytokine in the implanted group, suggesting that switching to CD8+ cells activation might play a role in the constant elimination of the antigen induced by the system of the present invention. In agreement with this difference, only the implanted mice, but not the immunized group switched immunoglobulin isotypes and produced IgG2a. The production of this isotype may be mediating the Th1/Th2 phenotype switch observed in the implanted group, and not found in the immunized mice.

It has been postulate that presentation of the Ag by APCs, is critical for induction of CTLs through MHC class I. It is therefore likely that the classical scheme of immunization failed to activate CD8+ cells and to generate the cytokine milieu and T cells required for proliferation of CTL and the destruction of target tissue. However, it is capable of activating CD4+T cells with an apparent bias for the Th2 subset, resulting in antibody formation against hFIX, primarily of the IgG1 subclass. Conversely, CASS was able to provide the necessary elements for both types of activation. These results might be of great importance in developing subunit vaccines against bacterial, viral and parasite infections, or tumors where both humoral and cellular immunity are crucial.

It is accepted that host exposure to excessive amounts of antigen for a prolonged period of time renders the host unresponsive by T-cell deletion. This event has been well documented for superantigens, non-cytopathic viruses and for proteins and peptides. It is also known that expression of large amounts of relevant antigen only in the periphery and not in lymphoid organs fails to induce a T cells response or to exhaust it. Our findings support the evidence that constant stimulation of the immune system by the system of the present invention favors T cell activation, and with the help of cytokines, enhances and prolongs T cell amplification and survival, thereby decreasing chances of T cell deletion or exhaustion.

Immunized mice developed neutralizing antibodies against autoantigens (FIG. 11). The same mechanism that enhances specific immune responses to foreign antigens can also lead to autoimmunity, posing a safety concern. Classical schemes of immunization provide an unabated stimulus for the production of signals that confuse the immune system, consequently facilitating the generation of autoantigen-recognizing lymphocytes clones and the excess production of specific neutralizing antibodies and regulator cells. The immunized mice in this study may have experienced a similar process, since they developed inhibitors causing acquired hemophilia B in these animals. In contrast, despite getting a better and longer immune response, mice treated with this system of the present invention did not show this complication. It is possible that activation of the immune response with a naturally folded protein may prevent cross-reactivity. In accordance with this aspect of the present invention, a novel concept in immunization based on continuous immune stimulation is described. It is fully contemplated that this aspect of the present invention will have applications in the design of novel vaccines against life-threatening diseases.

### EXAMPLE III

### Use of Encapsulated Recombinant Cells in Cancer Therapy

The present study was designed to evaluate the immunogenicity of an encapsulated genetically modified myoblast cell line as a potential cancer vaccine and/or therapy. JPW01 is a myoblast cell line developed in accordance with the present invention according to a protocol discussed further hereinbelow. A genetically modified cell of the present invention may be modified according to any method known in the art, including, but not limited to transfection, transduction or electroporation to elicit an immune response. When injected into C57BL/6 mice, JPW01 cells were found to cause tumors. Furthermore, C57BL/6 mice implanted with JPW01 encapsulated cells were also found to develop tumors due to the leakage of a small fraction of the cells from microcapsules. According to the present invention, these JPW01 cells were subsequently genetically modified to express a strong antigen, such as a bacterial LacZ gene product, for example. C57BL/6 mice were implanted intraperitoneally with the encapsulated JPW01/LacZ cells. In contrast with the mice implanted with encapsulated unmodified JPW01 cells, these mice did not develop tumors. Furthermore, as discussed in detail hereinbelow, the presence of encapsulated JPW01/LacZ cells in C57BL/6 mice appeared to provide an immunogenic protection against exposure to tumorigenic JPW01 cells, herein referred to as a anti-tumorgenic response. Thus, indicating that the immune response generated against LacZ conferred an immunity against the tumorgenicity of JPW01 cells.

It should be fully understood that suitable cells can be modified in accordance with the present invention to express a variety of different antigen(s) capable of eliciting an antigenic response against the source of a disease and/infection of interest, such as eliciting an antigenic response against a chronic infectious or neoplastic disease. This aspect of the present invention is herein exemplified with respect to cancer and immunogenicity thereto but is not limited thereto.

Materials and Methods

Isolation of Primary Mouse Myoblasts

Primary myoblasts were obtained using a protocol adapted from that described by Rando and Blau. Journal of Cell Biology 125(6) 1275-1287, 1994 which is herein incorporated by reference. The muscle biopsy is washed in cold PBS and placed in a 60 mm tissue culture dish. Any visible fat and connective tissue is removed and the remaining tissue is minced with a sterile scalpel until it reaches the consistency of a fine paste. This slurry is collected in a 15 ml tube and digested in a solution of collagenase and trypsin at 37° C. for 30 minutes, with vigorous mixing of the tube every 5 minutes. The now dissociated cells are pelleted by centrifugation, washed with serum free media, and centrifuged again. The cell pellet is re-suspended in 3 ml growth media (Ham's F10 supplemented with 20% FBS, 1% penicillin, 1% streptomycin and 2.5% ng/ml basic fibroblast growth factor) and pre-plated on a 60 mm tissue culture dish for 24 hours. Following this incubation, the supernatant is harvested and placed in a 60 mm collagen-coated tissue culture dish. The “pre-plating” of the cells allows for enrichment of myoblasts, as contaminating fibroblasts will adhere preferentially to the uncoated dish. Growth of the cells is monitored and the media is replaced with pre-warmed growth media every 48 hours. Cells are not allowed to reach confluence and each time they are split they are pre-plated on uncoated dishes for 20 minutes.

Cell Encapsulation

The JPW01 cells were encapsulated according to a procedure as described in Hortelano et al., 1996, which is herein incorporated by reference, with minor modifications. Briefly, a suspension of cells was mixed with 2% potassium alginate (Kelmar; Kelco, Chicago, Ill.) in a syringe and extruded as droplets through a 27-gauge needle with a syringe pump (39.3 ml/hr). The gelled droplets were collected in a 1.1% Ca Cl₂ solution. The outer alginate layer was cross-linked with poly-L-lysine (PLL; Sigma, St. Louis, Mo.) with a molecular weight in the 15,000-30,000 range for 6 min, and then coated with alginate. The core of unpolymerized alginate was dissolved with sodium citrate for 6 min to yield microcapsules with the cells suspended within. All the above washing procedures were performed at 4-10° C. The microcapsules were cultured in vitro under standard tissue culture conditions until implanted into mice.

Results

Primary myoblasts causing tumours (JPW01), when transduced to express LacZ gene, did not cause tumors in implanted mice, while resisting the challenge of direct injection of free and unmodified (and thus tumorigenic) JPW01 cells. As illustrated in FIG. 12 C57BL/6 mice implanted intraperitoneally with encapsulated JPW01/LacZ cells sustained exposure to untransfected JPW01 cells while all of the control mice, not previously implanted with microcapsules died within 60 days of exposure. In particular, 100% of the mice implanted with encapsulated JPW01/LacZ cells were still alive at 120 days post-treatment. For clarity, the untransfected JPW01 cells were not modified to secrete LacZ. This finding indicates that the immune response generated against LacZ also generated immunity against tumorigenic cell line JPW01.

Discussion

According to this aspect of the present invention, an improved anti-tumorigenic microcapsule or otherwise suitable implantable device, is provided for eliciting a tumor-specific immunity to cells implanted therewith. Cells of the present invention may be prepared for implantation by microencapsulation or any other suitable method known in the art so as to provide long-term release of an antigen of interest and/or immune stimulation in vivo. The improved anti-tumorigenic microcapsule of the present invention provides a distinct advantage in that it has been favorably modified so as not to cause tumors in mammals implanted therewith. Furthermore, this microcapsule is shown to provide a long-term, sustained immunogenic response having anti-tumorigenic capabilities. It is fully contemplated that a cell can be modified to express any suitable antigen when encapsulated and implanted in vivo in accordance with the present invention. In accordance with one embodiment of the present invention, a cell of interest is modified to express a strong antigen, such as LacZ, for example. Furthermore, a cell of interest may be modified to express one or more antigens for eliciting a desired immune response in vivo. Although this aspect of the present invention is exemplified in accordance with cancer, it is fully contemplated that the applications of the present invention extend to include vaccination and treatment of other diseases, including infectious diseases such as HIV for example. Accordingly, this aspect of the present invention has application in the generation of vaccines for cancer and infectious disease and for treatments thereof.

All references cited herein are incorporated herein by reference to the same extent as if each individual publication was specifically and individually indicated to be incorporated by reference in its entirety for all purposes.

The embodiments of the invention described above are intended to be exemplary only. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

I/We claim:

1. A process for inducing an immune response in a host, wherein said process comprises the steps of:
(a) enclosing genetically engineered antigen-producing cells comprising one or more transgene encoding antigen, into an immunoisolating implantable device to provide encapsulated antigen-producing cells;
(b) introducing the encapsulated antigen-producing cells in said host;
(c) production of the antigen product;
(d) bi-directional passage of the produced antigen product through the pores of said microcapsules, with preclusion of the passage of the antigen-producing cells therethrough;
(e) delivery of a continuous infusion of antigen to said host;
(f) activation of the immune system in response to said antigen.

2. The process of claim 1, wherein said antigen-producing cells comprise a myoblast cell.

3. The process of claim 2, wherein said myoblast cell comprises C2C12 and/or JPW01 mouse myoblast cells.

4. The process of claim 1, wherein said antigen-producing cells comprise mammalian cells.

5. The process of claim 1, wherein said antigen-producing cells comprise human cells.

6. The process of claim 1, wherein said antigen product is a weak immunogenic antigen.

7. The process of claim 1, wherein said antigen-producing cells may produce one or more different transgene antigens.

8. The process of claim 1, wherein said implantable device comprises biocompatible microcapsules.

9. The process of claim 1, wherein said implantable device comprises an immunoisolating implantable device.

10. The process of claim 1, wherein said implantable device comprises a hydrogel material.

11. The process of claim 1, wherein said b implantable device comprise alginate-poly-L-lysine microcapsules.

12. The process of claim 1, wherein said implantable device are non-antigenic.

13. The process of claim 1, wherein said implantable device comprise pores.

14. The process of claim 1, wherein said pores allow the bi-directional free flow of the produced antigen therethrough, but do not allow the free flow of the antigen-producing cells.

15. The process of claim 1, wherein the pores comprise a pore size that allow the bi-directional free-flow of molecules with a molecular weight up to that of the produced antigen.

16. The process of claim 1, wherein the pore size allows for the bi-directional free-flow of antigen with a molecular weight of about 300,000 daltons.

17. The process of claim 1, wherein the encapsulated antigen-producing cells are implanted intraperitoneally by injection.

18. The process of claim 1, wherein the encapsulated antigen-producing cells are implanted by subcutaneous injection, intramuscular injection, or any other form of administration that would allow for the implantation of viable an implantable device comprising antigen-producing cells in a host.

19. The process of claim 1, wherein the continuous infusion of antigen provides a continuous supply of antigen to said host.

20. The process of claim 1, wherein the encapsulated antigen-producing cells produce a continuous supply of active antigen to said host.

21. The process of claim 1, wherein the activation of the immune system comprises the sustained activation of both humoral and cellular immunity.

22. The process of claim 4, wherein the activation of the immune system comprises the sustained activation of both humoral and cellular immunity.

23. The process of claim 1, wherein the activation of the immune system comprises the activation of both humoral and cellular immunity against the antigen, comprising the production of antibodies against the transgene antigen and the activation of cytotoxic T lymphocyte (CTL) cells.

24. A method of immunizing a host, wherein said method comprises:
(a) introducing an immunoisolating implantable device enclosing antigen-producing cells comprising one or more transgenes encoding antigen, in vivo into said host;
(b) production of transgene antigen product by said antigen-producing cells;
(c) bi-directional passage of the produced antigen product through the pores of said implantable device, with preclusion of the passage of the antigen-producing cells therethrough;
(d) delivery of a continuous infusion of antigen to said host;
(e) sustained activation of the immune system in response to said antigen.

25. The method of claim 24, wherein said immunoisolating implantable device comprise non-antigenic biocompatible microcapsules.

26. The method of claim 24, wherein said method comprises immunizing a host with said antigen product.

27. The method of claim 24, wherein said method comprises immunizing a host with a weakly immunogenic antigen.

28. The method of claim 24, wherein said method comprises immunizing a host against infectious diseases.

29. The use of immunoisolating implantable devices enclosing genetically engineered antigen-producing cells for eliciting immune responses against an antigen in a host.

30. The use of immunoisolating implantable devices enclosing genetically engineered antigen-producing cells as a vaccine.

31. The use of immunoisolating implantable devices enclosing genetically engineered antigen-producing cells comprising one or more transgene to one or more antigen to immunize a host against said antigen.

32. The use of claim 31, wherein said antigen is a weak immunogenic antigen.

33. A method of immunizing a host, comprising administering to said host an appropriate amount of non-antigenic implantable devices enclosing genetically engineered antigen-producing cells for producing a continuous infusion of antigen to said host to elicit the activation of the immune system against said antigen.

34. A method of claim 33, wherein said non-antigenic implantable device comprise biocompatible microcapsules.

35. A method of claim 33, wherein said genetically engineered antigen-producing cells comprise myoblast cells, or C2C12 mouse myoblasts cells, or JPW01 mouse myoblast cells or mammalian cells, or human cells.

36. A method of claim 33, wherein said activation of the immune system comprises the activation of the humoral and cellular responses of the immune system against said antigen.

37. A method of vaccinating a host against infectious agents, comprising administering to said host an appropriate amount of a non-antigenic implantable devices enclosing genetically engineered antigen-producing cells for producing a continuous infusion of antigen to said host to elicit the sustained and continuous activation of the humoral and cellular responses of the immune system against said antigen.

38. A method of claim 37, wherein said host is a mammal, and more preferably a human.

39. A vaccine comprising an appropriate amount of non-antigenic implantable devices enclosing genetically engineered antigen-producing cells for producing a continuous infusion of antigen to said host to elicit the activation of the immune system in a complete and sustained manner against said antigen.

40. A vaccine of claim 39, wherein said genetically engineered antigen-producing cells comprises myoblast cells, or C2C12 mouse myoblasts cells, or JPW01 myoblast cells or mammalian cells, or human cells.

41. A method of inducing an immune response in a host, wherein said method comprises the steps of:
(a) enclosing genetically engineered antigen-producing cells, comprising one or more transgene encoding antigen, into an immunoisolating implantable device to provide encapsulated antigen-producing cells;
(b) introducing the encapsulated antigen-producing cells in said host;
(c) production of one or more transgene antigen product in the enclosed cell;
(d) bi-directional passage of the produced antigen product through the pores of said microcapsules, with preclusion of the passage of the antigen-producing cells therethrough;
(e) delivery of a continuous infusion of antigen to said host;
(f) activation of the immune system in response to said antigen.

42. The process of claim 1, wherein said encapsulated antigen-producing cells may be an autologous, syngeneic, allogeneic or xenogeneic cell with respect to said host.

43. The method of claim 41 wherein said immune response is anti-tumorigenic.

44. The process of claim 1, wherein said immune response is anti-tumorigenic.

45. The method of claim 24, wherein said method comprises immunizing a host with a anti-tumorigenic antigen.

46. A method of claim 33, wherein said genetically engineered antigen-producing cells comprise myoblast cells, or C2C12 mouse myoblasts cells, or JPW01 mouse myoblast cells or mammalian cells, or human cells.

47. The method of claim 46 wherein said genetically engineered antigen-producing cells elicit an anti-tumorigenic immune response.

48. A method of vaccinating a host against cancer, comprising administering to said host an appropriate amount of a non-antigenic implantable device(s) enclosing genetically engineered antigen-producing cells for producing a continuous infusion of antigen to said host to elicit a sustained and continuous activation of the humoral and cellular responses of the immune system against said antigen; wherein said responses of the immune system are anti-tumorigenic.

49. A method of treating a mammal suffering from cancer, said method comprising administering to said mammal an appropriate amount of a non-antigenic implantable device(s) enclosing genetically engineered antigen-producing cells for producing a continuous infusion of antigen to said mammal to elicit a sustained and continuous activation of the humoral and/or cellular responses of the immune system against said genetically engineered antigen; wherein said response(s) of the immune system are anti-tumorigenic.

50. The method of claim 48, wherein said genetically engineered antigen-producing cells comprise myoblast cells, or C2C12 mouse myoblasts cells, or JPW01 mouse myoblast cells or mammalian cells, or human cells.

51. The method of claim 50 wherein said genetically engineered antigen-producing cells are genetically modified to express a strong antigen capable of eliciting an anti-tumorigenic response.

52. The method of claim 49, wherein said genetically engineered antigen-producing cells comprise myoblast cells, or C2C12 mouse myoblasts cells, or JPW01 mouse myoblast cells or mammalian cells, or human cells.

53. The method of claim 52 wherein said genetically engineered antigen-producing cells are genetically modified to express a strong antigen capable of eliciting an anti-tumorigenic response.

54. The use of immunoisolating implantable devices enclosing genetically engineered antigen-producing cells for eliciting immune responses against a tumor antigen in a host.

55. The use of immunoisolating implantable devices enclosing genetically engineered antigen-producing cells as, a anti-cancer vaccine.

56. The use of immunoisolating implantable devices enclosing antigen-producing cells genetically engineered to include genetic material coding for one or more antigen to immunize a host against said antigen.

57. The use of claim 56 wherein said antigen elicits an anti-tumorigenic immune response in vivo.

58. A process for introducing an antigen in a host, wherein said process comprises the steps of:
(a) encapsulating genetically engineered antigen-producing cells into an immunoisolating, implantable non-antigenic microcapsule, wherein the genetically engineered cells comprise one or more transgenes encoding an antigen, wherein the microcapsule comprises pores capable of allowing bi-directional passage of the produced antigen product through the pores, with preclusion of the passage of the antigen-producing cells therethrough; and
(b) introducing the antigen-producing cell-comprising devices into said host.
